(19) United States
(12) Patent Application Publication
(10) Pub. No.: US 2025/0297221 A1
(43) Pub. Date: Sep. 25, 2025

(54) GENETICALLY-MODIFIED PLURIPOTENT STEM CELLS AND DERIVED NATURAL KILLER CELLS AND METHODS FOR PRODUCING THE SAME

(71) Applicant: NUWACELL BIOTECHNOLOGIES CO., LTD., Hefei, Anhui (CN)

(72) Inventors: Junying YU, Hefei, Anhui (CN); Tao ZHOU, Hefei, Anhui (CN); Changqing TIAN, Hefei, Anhui (CN); Jian ZHANG, Hefei, Anhui (CN); Wentao SHAO, Hefei, Anhui (CN); Ying ZHANG, Hefei, Anhui (CN)

(73) Assignee: NUWACELL BIOTECHNOLOGIES CO., LTD., Hefei, Anhui (CN)

(21) Appl. No.: 18/260,406

(22) PCT Filed: Mar. 28, 2023
(86) PCT No.: PCT/CN2023/084233

§ 371 Date: Jul. 5, 2023

(51) Int. Cl. C12N 5/0783; C07K 14/54; C07K 14/735; C12N 5/074; C12N 9/22; C12N 15/11; C12N 15/85

(52) U.S. Cl. CPC C12N 5/0646; C07K 14/5443; C07K 14/70535; C12N 5/0696; C12N 9/22; C12N 15/111; C12N 15/85; C12N 2310/20; C12N 2506/45; C12N 2510/00

(57) ABSTRACT

Provided herein are genetically-modified cells such as pluripotent stem cells and derived NK cells and methods for producing the same. The gene editing strategy of the present disclosure can achieve higher expression of the transgene.

### TECHNICAL FIELD

This disclosure generally relates to genetically-modified pluripotent stem cells (PSCs) and their derivative natural killer (NK) cells and methods for producing the same.

### INCORPORATION BY REFERENCE OF SEQUENCE LISTING

The Substitute Sequence Listing in an XML file, named as 42194_SubstituteSequenceListing.xml of 126,976 bytes, created on Jan. 6, 2025, and submitted to the United States Patent and Trademark Office, via Patent Center, is incorporated herein by reference.

### BACKGROUND

Adoptive cell therapy generally involves administration of immune cells to patients having cancer, tumors, or infections. As one of the lymphocytes that play an important role in immunoreaction, NK cells have grown as one of the most promising agents for adoptive cell therapy. In particular, pluripotent stem cell (PSC)-derived NK cells (also referred to as induced NK or iNK cells) have attracted wide attention due to their strong cytotoxicity, high safety and easy cell availability. Notwithstanding, the use of iNK cells for cell therapy remains to be challenging and has unmet needs for improvement. For example, current iNK cells need to have the cell functions such as cytotoxicity enhanced to satisfy various clinical applications. In order to enhance the function or cytotoxicity of the iNK cells for cell therapy, various gene editing strategies have been developed to produce the genetically modified or engineered iNK cells. However, current gene editing strategies for iNK cells often encounter some serious problems such as gene silencing or reduced gene expression during the production (e.g., differentiation) of the iNK cells.

Accordingly, there remains a need for improved genome editing strategy for genetically modified or engineered iNK cells.

### SUMMARY

Gene silencing has always been an unavoidable problem for gene therapy and cell therapy associated with gene editing. The reasons for gene silencing are diversified and complicated, and may be associated with, for example, the change of epigenetics (DNA methylation and histone modification) for some specific sequences such as repeat sequences, virus sequences and bacterium-associated sequences. The present inventors have found that it is crucial for preventing the gene silencing to select a suitable target site for gene editing. Many gene loci in the PSCs are in the silencing state due to high methylation and the expression of target gene integrated at these loci cannot be observed or detected. Further, the gene editing at some loci can adversely affect the pluripotency and differentiation potential of the PSCs. In addition, some loci may be silenced and thus give rise to the reduction and loss in the expression of target gene during the differentiation of PSCs into functional cells such as iNK cells. Therefore, there remains a need to systematically investigate various target sites for gene editing in order to achieve the stable expression of exogenous gene while maintaining the characteristics and the differentiation potential of PSCs.

In one aspect, there is provided genetically-modified pluripotent stem cells (PSCs), comprising an expression cassette integrated at a selected locus of the genome of PSCs, the expression cassette comprising one or more exogenous polynucleotides of interest, and one or more promoters operably linked to the one or more exogenous polynucleotides of interest, wherein the selected locus is CISH and/or Rosa26, and wherein the genetically-modified PSCs are capable of differentiation into genetically-modified iNK cells having higher expression level and uniformity for the one or more exogenous polynucleotides of interest as compared with the PSCs genetically modified under the same conditions except for the integration at a locus other than CISH and/or Rosa26.

In another aspect, there is provided genetically-modified iNK cells, comprising an expression cassette integrated at a selected locus, the expression cassette comprising one or more exogenous polynucleotides of interest, and one or more promoters operably linked to the one or more exogenous polynucleotides of interest, wherein the selected locus is CISH and/or Rosa26, and wherein the genetically-modified iNK cells have higher expression level and uniformity for the one or more exogenous polynucleotides of interest as compared with the iNK cells genetically modified under the same conditions except for the integration at a locus other than CISH and/or Rosa26.

In a further aspect, there is provided a method for producing genetically-modified pluripotent stem cells (PSCs), comprising: introducing into PSCs a construct comprising a site-specific endonuclease capable of introducing a double strand break at a selected locus of the genome of the PSCs and a construct comprising an expression cassette comprising one or more exogenous polynucleotides of interest and one or more promoters operably linked to the one or more exogenous polynucleotides of interest and a pair of homology arms specific to the selected locus and flanking the expression cassette; and integrating the expression cassette comprising the one or more exogenous polynucleotides of interest and the one or more promoters into the genome of the PSCs at the selected locus via homologous recombination by the endonuclease to obtain the genetically-modified PSCs, wherein the selected locus is CISH and/or Rosa26; wherein the genetically-modified PSCs are capable of differentiation into genetically-modified iNK cells having higher expression level and uniformity for the one or more exogenous polynucleotides of interest as compared with the PSCs genetically modified under the same conditions except for the integration at a locus other than CISH and/or Rosa26.

In still another aspect, there is provided a method for producing genetically-modified iNK cells, comprising producing genetically-modified PSCs according to the method described herein and then differentiating the genetically-modified PSCs into NK cells, thereby producing the genetically-modified iNK cells, wherein the genetically-modified iNK cells have higher expression level and uniformity for the one or more exogenous polynucleotides of interest as compared with the iNK cells genetically modified under the same conditions except for the integration at a locus other than CISH and/or Rosa26.

Various objects and advantages of various aspects as provided herein will become apparent from the following description taken in conjunction with the accompanying drawings wherein are set forth, by way of illustration and example, certain embodiments of the present disclosure.

### DESCRIPTION OF DRAWINGS

FIG. 1 is a schematic diagram of Knock-in of Antares2 at different loci, Rosa26, CISH, AAVS1, CD38 and NKG2A, in the genome of human induced pluripotent stem cells (hiPSCs) according to the Example 1 of the present disclosure.

FIG. 2A shows the cell percentages (CD56+%) of the iNK cells engineered at different loci, Rosa26, CISH, AAVS1, CD38 and NKG2A, according to the Example 1 of the present disclosure, where WT represents wild type.

FIG. 2B shows the results of luciferase assay for the expression of Antares2 in both hiPSCs and iNK cells engineered at different loci, Rosa26, CISH, AAVS1, CD38 and NKG2A, according to the Example 1 of the present disclosure.

FIG. 2C shows the results of flow cytometry analysis for the expression of Antares2 in both hiPSCs and iNK cells engineered at different loci, Rosa26, CISH, AAVS1, CD38 and NKG2A, according to the Example 1 of the present disclosure, where WT represents wild type.

FIG. 3A shows the cell percentages (CD16+%) of the NK92 cells randomly integrated with different expression cassettes after the expansion in the presence or absence of exogenous IL-15 according to the Example 2 of the present disclosure, where #1-#8 represent the expression cassettes CD16A-T2A-NeoIL2, CD16A-T2A-IL15RAsu, CD16A-T2A-IL15RLI, CD16A-T2A-IL15ILR, CD16A-T2A-IL15-TPA, CD16A-T2A-mbIL15, CD16A-T2A-mbIL15RLI, and CD16A-T2A-mbIL21-mbIL15, respectively.

FIG. 3B shows the total cell numbers (CD16+ cells) of the NK92 cells randomly integrated with different expression cassettes after the expansion in the presence or absence of exogenous IL-15 according to the Example 2 of the present disclosure, where #1-#8 represent the expression cassettes CD16A-T2A-NeoIL2, CD16A-T2A-IL15RAsu, CD16A-T2A-IL15RLI, CD16A-T2A-IL15ILR, CD16A-T2A-IL15-TPA, CD16A-T2A-mbIL15, CD16A-T2A-mbIL15RLI, and CD16A-T2A-mbIL21-mbIL15, respectively.

FIG. 3C shows the results of flow cytometry analysis for the expression of CD16A or CD64/CD16A in the NK92 cells randomly integrated with different expression cassettes according to the Example 3 of the present disclosure, where #1-#8 represent the expression cassettes CD16A-T2A-NeoIL2, CD16A-T2A-IL15-TPA, CD16A-T2A-IL15RLI, CD16A-T2A-mbIL15RLI, CD64/16A-T2A-NeoIL2, CD64/16A-T2A-IL15-TPA, CD64/16A-T2A-IL15RLI, and CD64/16A-T2A-mbIL15RLI, respectively.

FIG. 3D shows the antibody-dependent cell-mediated cytotoxicity (ADCC) function of the NK92 cells randomly integrated with different expression cassettes against SK-OV-2 cells according to the Example 3 of the present disclosure, where #1-#8 represent the expression cassettes CD16A-T2A-NeoIL2, CD16A-T2A-IL15-TPA, CD16A-T2A-IL15RLI, CD16A-T2A-mbIL15RLI, CD64/16A-T2A-NeoIL2, CD64/16A-T2A-IL15-TPA, CD64/16A-T2A-IL15RLI, and CD64/16A-T2A-mbIL15RLI, respectively.

FIG. 3E shows the ADCC function of the NK92 cells randomly integrated with different expression cassettes against Raji cells according to the Example 3 of the present disclosure, where #1-#8 represent the expression cassettes CD16A-T2A-NeoIL2, CD16A-T2A-IL15-TPA, CD16A-T2A-IL15RLI, CD16A-T2A-mbIL15RLI, CD64/16A-T2A-NeoIL2, CD64/16A-T2A-IL15-TPA, CD64/16A-T2A-IL15RLI, and CD64/16A-T2A-mbIL15RLI, respectively.

FIG. 4 shows the results of flow cytometry analysis for the expression of GFP in the hiPSCs and iNK cells engineered with different promoters (EF1α, CMV and CLP) and the cell percentages (CD56+%) of the iNK cells according to the Example 4 of the present disclosure.

FIG. 5 shows the effects of the prolonged expansion of the engineered hiPSCs on the CD64 expression and the cell percentage (CD56+%) of the derivative iNK cells according to the Example 5 of the present disclosure.

FIG. 6A shows the effect of different versions of UCOE anti-silencing element on the expression of membrane-bound anti-PD-L1 nano-antibody (mbPDL1 Nb) randomly inserted in the hiPSCs according to the Example 6 of the present disclosure.

FIG. 6B shows the comparison of the anti-silencing effects of the two UCOE elements, 1550F and SRF6-3F, in the hiPSCs randomly inserted with mbPDL1 Nb after the prolonged expansion according to the Example 7 of the present disclosure.

FIG. 6C shows the effect of cHS4 insulator on the expression of mbPDL1 Nb in the hiPSCs randomly inserted with mbPDL1 Nb after the prolonged expansion according to the Example 8 of the present disclosure.

FIG. 7A shows the results of the flow cytometry analysis for the expression of GFP driven by different promoters (EF1α and CMV) with or without 1550F UCOE element in the randomly inserted iNK cells according to the Example 9 of the present disclosure.

FIG. 7B shows the results of the analysis of the mean GFP fluorescence for the expression of GFP driven by different promoters (EF1α and CMV) with or without 1550F UCOE element in the randomly inserted iNK cells according to the Example 9 of the present disclosure.

FIG. 8 shows the anti-silencing effect of 1550F UCOE element on the expression of CD64/CD16A in the iNK cells differentiated from the engineered hiPSCs after the prolonged expansion according to the Example 10 of the present disclosure.

FIG. 9 shows the anti-silencing effect of the combinations of different promoters (EF1α and CMV) with 1550F UCOE element on the expression of CD64/CD16A in the immature (D24 iNK) and mature (D32 iNK) engineered iNK cells according to the Example 11 of the present disclosure, where WT represents wild type.

FIG. 10A shows the results of flow cytometry analysis for the expression of CD16A inserted in the engineered hiPSCs according to the Example 12 of the present disclosure, where WT iPSC is used as a control.

FIG. 10B shows the results of flow cytometry analysis for the expression of CD16A inserted in the engineered iNK cells according to the Example 12 of the present disclosure, where WT iNK is used as a control.

### DETAILED DESCRIPTION

It is to be appreciated that certain aspects, modes, embodiments, variations and features of the present disclosure are described below in various levels of detail in order to provide a substantial understanding of the present technology.

Reference throughout this specification to “first,” “second,” “third,” “fourth,” “fifth,” “sixth,” etc. does not mean the order or sequence of the feature, structure or characteristic described in connection with the reference and can be used only for the purpose of distinction.

Reference throughout this specification to “a first aspect,” “a second aspect,” “a third aspect,” “a fourth aspect,” “a fifth aspect,” “a sixth aspect,” etc. means that a particular feature, structure or characteristic described in connection with the aspect is included in at least one or more aspects of the present disclosure. Also, the particular feature(s), structure(s), characteristic(s) or embodiment(s) in one aspect may be combined with those in one or more other aspects in any suitable manner.

Reference throughout this specification to “one embodiment,” “some embodiments,” “a preferred embodiment(s),” “certain embodiments” or “a certain embodiment(s)” means that a particular feature, structure or characteristic described in connection with the embodiment(s) is included in at least one or more embodiments of the present disclosure. Also, the particular feature(s), structure(s), or characteristic(s) in one embodiment may be combined with those in one or more other embodiments in any suitable manner.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

Unless otherwise specified, “a” or “an” means “one or more.”

Unless otherwise specified, the use of the alternative (e.g., “and/or”) should be understood to mean either one, both, or any combination thereof of the alternatives.

As used herein, “about” means plus or minus 10%, or plus or minus 5%, or plus or minus 4%, or plus or minus 3%, or plus or minus 2%, or plus or minus 1%, as well as the specified number.

As used herein, the term “substantially” or “essentially” refers to a quantity, level, value, number, frequency, percentage, dimension, size, amount, weight or length that is about or at least 90%, 91%, 92%, 93%, 94%, 95%, 96%, 97%, 98%, or 99% or higher compared to a reference quantity, level, value, number, frequency, percentage, dimension, size, amount, weight or length. In some embodiments, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of). Further, in each instance herein any of the terms “comprising,” “consisting essentially of,” and “consisting of” may be replaced with either of the other two terms.

As used herein, the term “pluripotent stem cells” (PSCs) refers to cells derived from the inner cell mass of the embryonic blastocyst. Pluripotent stem cells can be pluripotent and give rise during development to all derivatives of the three primary germ layers: ectoderm, endoderm and mesoderm. Pluripotent stem cells can be of human origin (e.g., human PSC or hPSC). Pluripotent stems cells can be induced pluripotent stem cells (iPSCs) or embryonic stem cells (ESCs). ESCs (e.g., hESCs) and iPSCs (e.g., hiPSCs) are known in the art and can be readily obtained using conventional methods, for example, those described in the existing technologies, or commercially available products. PSCs obtained following various types of genetic engineering, such as genomic locus-specific transgene knock-in can also be used herein.

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature. Suitable methods for the generation of iPSCs from somatic or multipotent stem cells are well known to those of skill in the art. For example, iPSCs may be reliably generated from somatic cells by conventional reprogramming technologies.

As used herein, the term “reprogramming” refer to a method of increasing the potency of a cell or dedifferentiating a cell to a less differentiated state. For example, a cell that has an increased cell potency can have more developmental plasticity (i.e., can differentiate into more cell types) compared to the same cell in the non-reprogrammed state. That is, a reprogrammed cell is one that is in a less differentiated state than the same cell in a non-reprogrammed state. “Reprogramming” can refer to dedifferentiating a somatic cell, or a multipotent stem cell, into a pluripotent stem cell, also referred to as an induced pluripotent stem cell, or iPSC.

As used herein, the term “embryonic stem cells” or, ESCs refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extraembryonic membranes or the placenta, i.e., are not totipotent. When used in the present disclosure, the embryonic stem cells or ESCs are obtained from commercially established human embryonic stem cell lines or human embryonic stem cells that have not been developed in vivo within 14 days of fertilization.

As used herein, the term “pluripotency” or “pluripotent” refers to a cell that has the developmental potential to differentiate into cells of all three germ layers (Ectoderm, mesoderm, and endoderm). Pluripotency can be determined, at least in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

As used herein, the term “genetically-modified pluripotent stem cells” (PSCs) refers to pluripotent stem cells which have been modified to comprise at least one exogenous gene in the genome. In the context of this disclosure, the genetically-modified PSCs can be used interchangeably with the genetically-engineered PSCs or gene-edited PSCs.

As used herein, “natural killer cells” or NK cells refers to lymphoid cells defined by its marker expression and function/activity. For example, in humans, NK cells expresses CD56. For example, such NK cells may be CD56+CD3− cells. NK cells may express variable levels of CD56. NK cells may comprise primary NK cells or induced NK (iNK) cells.

As used herein, “primary NK cells” refers to naturally occurring natural killer cells which can be sourced from, for example, blood (e.g., cord blood or peripheral blood collected by apheresis), bone marrow or frozen primary NK cells (e.g., commercially available). Examples of primary NK cells comprises PBNK (peripheral blood-derived NK) and CBNK (cord blood-derived NK) cells.

As used herein, the term “INK cells” refers to natural killer cells derived from pluripotent stem cells (e.g., hPSCs). The iNK cells may comprise immature or mature iNK cells.

As used herein, the term “immature iNK cells” refers to natural killer cells which are directly differentiated from pluripotent cells (e.g., hPSCs) and have been not subjected to the expansion and maturation. The immature iNK cells have lower expression for specific markers such as CD56 and have lower cytokine-releasing function and cytotoxity as compared with mature iNK cells.

As used herein, the term “mature iNK cells” or “matured iNK cells” refers to natural killer cells which are differentiated from pluripotent cells (e.g., hPSCs) and have been subjected to the expansion and maturation. The mature iNK cells have higher expression for specific markers such as CD56 and have the cytokine-releasing function and cytotoxity similarly to primary NK cells.

As used herein, the term “genetically-modified iNK cells” refers to iNK cells which have been modified to comprise at least one exogenous gene in the genome. In the context of this disclosure, the genetically-modified iNK cells can be used interchangeably with the genetically-engineered iNK cells or gene-edited iNK cells.

As used herein, the term “differentiation” refers to the process by which an unspecialized (“uncommitted”) or less specialized cell acquires the features of a specialized cell such as, for example, a blood cell or an immune cell. In certain embodiments, a differentiated or differentiation-induced cell is one that has taken on a more specialized (“committed”) position within the lineage of a cell. For example, a human Pluripotent Stem Cell (hPSCs) can be differentiated into various more differentiated cell types, for example, a neural or a hematopoietic progenitor cell, a lymphocyte, a cardiomyocyte, an immune cell (e.g., a Natural Killer cell), and other cell types, upon treatment with suitable differentiation factors in the cell culture medium. In certain embodiments, the term “committed” is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

As used herein the term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells. In certain embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells, and anergic AN1/T3 cell populations.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

As used herein, the term “expression cassette” refers to the complete elements required to express a gene, including an operably linked promoter and gene coding sequence.

As used herein, the term “coding sequence” refers to that portion of a nucleic acid sequence which directly specifies the amino acid sequence of its protein product. The boundaries of the coding sequence are generally determined by the ribosome binding site (for prokaryotic cells) immediately upstream of the 5′ open reading frame of the mRNA and the transcription termination sequence immediately downstream of the 3′ open reading frame of the mRNA.

As used herein, the term “gene(s) of interest” or “polynucleotide(s) of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode a mRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “exogenous” in intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell.

As used herein, the term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, the term “operably linked” or “operatively linked” refers to the association of nucleic acid sequences on a single nucleic acid fragment so that the function of one is affected by the other. For example, a promoter is operably linked with a coding sequence or functional RNA when it is capable of affecting the expression of that coding sequence or functional RNA (i.e., the coding sequence or functional RNA is under the transcriptional control of the promoter). Coding sequences can be operably linked to regulatory sequences in sense or antisense orientation.

By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

As used herein, the term “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

As used herein, the term “continuous expansion” refers to the long-term expansion of cells where the cells are passaged for multiple passages. In the context of this disclosure, the continuous expansion can be used interchangeably with the prolonged expansion.

As used herein, the term “effective amount” refers to a quantity of an agent sufficient to achieve a beneficial or desired result upon administration. The amount of an agent administered to the subject can depend on the characteristics of the individual, such as general health, age, sex, body weight, effective concentration of the cells (e.g., iNK cells) administered, and tolerance to drugs. The skilled artisan will be able to determine appropriate dosages depending on these and other factors. An effective amount can be administered to a subject in one or more doses.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular medical intervention, or from whom cells are harvested). In certain embodiments, the individual, patient or subject is a human.

As used herein, the terms “treatment,” “treat,” and “treating” refer to a clinical intervention aimed to reverse, alleviate, delay the onset of, or inhibit the progress, ameliorate, reduce severity of, prevent or delay the recurrence of a disease, disorder, and/or condition or one or more symptoms thereof, and/or improve one or more symptoms of a disease, disorder, and/or condition as described herein. Treatment, e.g., in the form of edited PSCs or iNK cells or a population of edited PSCs or iNK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, treatment may be administered to a susceptible individual prior to the onset of symptoms (e.g., in light of genetic or other susceptibility factors). Treatment may also be continued after symptoms have resolved, for example to prevent or delay their recurrence. Treatment can result in improvement and/or resolution of one or more symptoms of a disease, disorder and/or condition.

As used herein, the terms “prevent,” “preventing,” and “prevention” refer to reducing the probability of developing a disease, disorder, or condition in a subject, who does not have, but is at risk of or susceptible to developing a disease, disorder, or condition.

### Genetically-Modified Cells

The gene editing strategy for obtaining engineered iNK cells from engineered PSCs requires that the developmental potential of the engineered PSCs in a directed differentiation is not adversely impacted by the engineered modality in the PSCs, and also that the engineered modality can be highly expressed to function as intended in the derived NK cells. The genetically-engineered PSCs and iNK cells as disclosed herein comprise one or several genetic modifications at selected specific gene locus in their genome. The above specific gene locus comprises Rosa 26 and/or CISH. The genetically-engineered PSCs generated by the targeted integration at Rosa 26 and/or CISH are capable of differentiation into NK cells having higher expression level and uniformity for the one or more exogenous polynucleotides of interest as compared with the PSCs genetically modified under the same conditions except for the integration at a locus other than CISH and/or Rosa26. Further, this strategy overcomes the current barrier in engineering primary NK cells sourced from peripheral blood, umbilical cord blood or any other donor tissues, as such cells are limited in supply and difficult to engineer, with engineering of such cells often lacking reproducibility and uniformity.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic modification”) enables insertion, deletion, and/or substitution at pre-selected sites in the genome. When an endogenous sequence is inserted, deleted, and/or replaced at the pre-selected site during targeted editing, an endogenous gene comprising the affected sequence can be knocked-out or knocked-down. Therefore, targeted editing may also be used to disrupt endogenous gene expression.

Similarly used herein is the term “targeted integration,” referring to a process involving insertion of one or more exogenous sequences, with or without deletion of an endogenous sequence at the insertion site. In comparison, random integration or gene editing (using transposon system, for example) are subject to position effects and silencing, producing their expression unreliable and unpredictable. In addition, random integration may activate protooncogene, which brings about safety issue.

In one aspect, there are provided genetically-modified pluripotent stem cells (PSCs), comprising an expression cassette integrated at a selected locus of the genome of PSCs, the expression cassette comprising one or more exogenous polynucleotides of interest, and one or more promoters operably linked to the one or more exogenous polynucleotides of interest, wherein the selected locus is CISH and/or Rosa26, and wherein the genetically-modified PSCs are capable of differentiation into genetically-modified iNK cells having higher expression level and uniformity for the one or more exogenous polynucleotides of interest as compared with the PSCs genetically modified under the same conditions except for the integration at a locus other than CISH and/or Rosa26.

In certain embodiments, the genetically-modified PSCs are capable of differentiation into the iNK cells at the efficiency of at least 90%, such as for example, 91% or more, 92% or more, 93% or more, 94% or more, or 95% or more. In certain embodiments, the genetically-modified PSCs are capable of differentiation into the iNK cells at the efficiency of at least 96%. In certain embodiments, the genetically-modified PSCs are capable of differentiation into the iNK cells at the efficiency of at least 97%. In certain embodiments, the genetically-modified PSCs are capable of differentiation into the iNK cells at the efficiency of at least 98%. In certain embodiments, the genetically-modified PSCs are capable of differentiation into the iNK cells at the efficiency of at least 99%. In certain embodiments, the genetically-modified PSCs are capable of differentiation into the iNK cells at the efficiency of 100%.

According to the present disclosure, the genetically-modified PSCs are capable of differentiation into genetically-modified iNK cells having at least 5 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 6, 7, 8, 9 or 10 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 11, 12, 13, 14 or 15 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 16, 17, 18, 19 or 20 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 21, 22, 23, 24 or 25 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 26, 27, 28, 29 or 30 times higher expression level for the one or more exogenous polynucleotides of interest than the PSCs genetically modified under the same conditions except for the integration at the locus other than CISH and/or Rosa26.

According to the present disclosure, the genetically-modified PSCs are capable of differentiation into genetically-modified iNK cells having at least one time higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least two times higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least three times higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least four times higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least five times higher expression uniformity for the one or more exogenous polynucleotides of interest than the PSCs genetically modified under the same conditions except for the integration at the locus other than CISH and/or Rosa26.

According to the present disclosure, the genetically-modified PSCs are capable of differentiation into iNK cells having at least 90%, such as for example, 91% or more, 92% or more, 93% or more, 94% or more, or 95% or more expression uniformity for the one or more exogenous polynucleotides of interest. In certain embodiments, the genetically-modified PSCs are capable of differentiation into iNK cells having at least 96% expression uniformity for the one or more exogenous polynucleotides of interest. In certain embodiments, the genetically-modified PSCs are capable of differentiation into iNK cells having at least 97% expression uniformity for the one or more exogenous polynucleotides of interest. In certain embodiments, the genetically-modified PSCs are capable of differentiation into iNK cells having at least 98% expression uniformity for the one or more exogenous polynucleotides of interest. In certain embodiments, the genetically-modified PSCs are capable of differentiation into iNK cells having at least 99% expression uniformity for the one or more exogenous polynucleotides of interest. In certain the genetically-modified PSCs are capable of embodiments, differentiation into iNK cells having 100% expression uniformity for the one or more exogenous polynucleotides of interest.

In some embodiments, the genetically-modified PSCs can differentiate into hematopoietic cell lineages or any other non-pluripotent cell types in vitro, wherein the derived non-pluripotent cells retain the functional genetic modification of the PSCs. In some embodiments, the genetically-modified PSC-derived cells include, but are not limited to, mesodermal cells, hemogenic endothelium (HE) cells, CD34 hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, and B cells, wherein these cells derived from the genetically-modified PSCs retain the functional genetic modification at the desired site.

In another aspect, there are provided genetically-modified iNK cells, comprising an expression cassette integrated at a selected locus, the expression cassette comprising one or more exogenous polynucleotides of interest, and one or more promoters operably linked to the one or more exogenous polynucleotides of interest, wherein the selected locus is CISH and/or Rosa26, and wherein the genetically-modified iNK cells have higher expression level and uniformity for the one or more exogenous polynucleotides of interest as compared with the iNK cells genetically modified under the same conditions except for the integration at a locus other than CISH and/or Rosa26.

According to the present disclosure, the one or more exogenous polynucleotides of interest can be highly uniformly expressed in the genetically-modified iNK cells described herein.

For example, the genetically-modified iNK cells have at least one time higher expression uniformity for the one or more exogenous polynucleotides of interest than the iNK cells genetically modified under the same conditions except for the integration at the locus other than CISH and/or Rosa26. In certain embodiments, the genetically-modified iNK cells have at least two times higher expression uniformity for the one or more exogenous polynucleotides of interest than the iNK cells genetically modified under the same conditions except for the integration at the locus other than CISH and/or Rosa26. In certain embodiments, the genetically-modified iNK cells have at least three times higher expression uniformity for the one or more exogenous polynucleotides of interest than the iNK cells genetically modified under the same conditions except for the integration at the locus other than CISH and/or Rosa26. In certain embodiments, the genetically-modified iNK cells have at least four times higher expression uniformity for the one or more exogenous polynucleotides of interest than the iNK cells genetically modified under the same conditions except for the integration at the locus other than CISH and/or Rosa26. In certain embodiments, the genetically-modified iNK cells have at least five times higher expression uniformity for the one or more exogenous polynucleotides of interest than the iNK cells genetically modified under the same conditions except for the integration at the locus other than CISH and/or Rosa26.

According to the present disclosure, the one or more exogenous polynucleotides of interest can be expressed in at least 90%, such as for example, 91% or more, 92% or more, 93% or more, 94% or more, or 95% or more of the genetically-modified iNK cells. In certain embodiments, the one or more exogenous polynucleotides of interest can be expressed in at least 96% of the genetically-modified iNK cells. In certain embodiments, the one or more exogenous polynucleotides of interest can be expressed in at least 97% of the genetically-modified iNK cells. In certain embodiments, the one or more exogenous polynucleotides of interest can be expressed in at least 98% of the genetically-modified iNK cells. In certain embodiments, the one or more exogenous polynucleotides of interest can be expressed in at least 99% of the genetically-modified iNK cells. In certain embodiments, the one or more exogenous polynucleotides of interest can be expressed in 100% of the genetically-modified iNK cells.

According to the present disclosure, the genetic modification at CISH does not influence the expression level and expression uniformity of the transgene in the final differentiated cells such as iNK cells, but does in the PSCs. Surprisingly, the expression of the transgene at CISH is silenced in pluripotent state but is recovered after iNK cell differentiation.

According to the present disclosure, the genetic modification at Rosa26 does not influence the expression level and expression uniformity of the transgene both in the PSCs and final differentiated cells such as iNK cells. In certain embodiments, the one or more exogenous polynucleotides of interest can be expressed in at least 50%, such as for example, 60% or more, or 70% or more of the genetically-modified PSCs. In certain embodiments, the one or more exogenous polynucleotides of interest can be expressed in at least 80%, such as for example, 81% or more, 82% or more, 83% or more, 84% or more, 85% or more, 86% or more, 87% or more, 88% or more, or 89% or more of the genetically-modified PSCs. In certain embodiments, the one or more exogenous polynucleotides of interest can be expressed in at least 90%, such as for example, 91% or more, 92% or more, 93% or more, 94% or more, 95% or more, 96% or more, 97% or more, 98% or more, 99% or more, or 100% of the genetically-modified PSCs.

### Gene Loci

In the present disclosure, CISH and Rosa26 are disclosed as the target site of genetic engineering in PSCs and its derivative iNK cells.

### CISH

Cytokine-inducible SH2-containing protein (CISH) is a protein that in humans is encoded by the CISH gene. CISH orthologs have been identified in most mammals with sequenced genomes. CISH controls T cell receptor (TCR) signaling, and variations of CISH with certain SNPs are associated with susceptibility to bacteremia, tuberculosis and malaria (see, e.g., Uchida K et al., March 1998, “Molecular cloning of CISH, chromosome assignment to 3p21.3, and analysis of expression in fetal and adult tissues”. Cytogenetics and Cell Genetics. 78 (3-4): 209-12; Yoshimura A, et al, June 1995, “A novel cytokine-inducible gene CIS encodes an SH2-containing protein that binds to tyrosine-phosphorylated interleukin 3 and erythropoietin receptors”. The EMBO Journal. 14 (12): 2816-26; and Khor C C et al., June 2010, “CISH and susceptibility to infectious diseases”. The New England Journal of Medicine. 362 (22): 2092-101).

Human CISH is located from position 50606489 to 50611774 in Chromosome 3 according to GRCh38.p14. It has 4 exons and 3 introns. The Gene ID for human CISH is 1154. More details regarding this gene can be found in NCBI database, which is incorporated herein by reference in its entirety.

Any site in the Human CISH locus can be chosen as the target site. For example, the target sequence of the CISH locus can be chosen from GenBank NM_013324.7. Examples of the target sequence of the CISH locus may comprise exon 1 (1 . . . 144), exon 2 (145 . . . 301), exon 3 (302 . . . 522) and exon 4 (523 . . . 2176).

### Rosa26

ROSA26 is a locus used for constitutive, ubiquitous gene expression in mice. It was firstly isolated in a gene-trap mutagenesis screen of embryonic stem cells (ESCs) (see, e.g., Friedrich, G; Soriano, P (1991). “promoter traps in embryonic stem cells: A genetic screen to identify and mutate developmental genes in mice”. Genes & Development. 5 (9): 1513-23). The human ROSA26 locus has been identified (Irion, Stefan; Luche, Hervé; Gadue, Paul; Fehling, Hans Joerg; Kennedy, Marion; Keller, Gordon (2007). “Identification and targeting of the ROSA26 locus in human embryonic stem cells”. Nature Biotechnology. 25 (12): 1477-82). ROSA stands for Reverse Orientation Splice Acceptor, named after the lentivirus genetrap vector.

More details regarding ROSA26 can be found in NCBI database, which is incorporated herein by reference in its entirety.

Any site in the Human Rosa26 locus can be chosen as the target site. For example, the target sequence of the Rosa26 locus can be chosen from Chr3: 9432781 . . . 9440914.

In certain embodiments, the locus other than CISH and/or Rosa26 is selected from AAVS1, CD38 and/or NKG2A.

### AAVS1

Adeno-associated virus integration site 1 (AAVS1) is a viral integration site that in humans is encoded by the AAVS1 gene located on chromosome 19 (see, e.g., Ward et al., Virology. 2012 Nov. 25; 433 (2): 356-66. doi: 10.1016/j.virol.2012.08.015. Epub 2012 Sep. 13; and Kotin et al., EMBO J. 1992 December; 11 (13): 5071-8. doi: 10.1002/j.1460-2075.1992.tb05614.x.). More details regarding AAVS1 locus can be found in NCBI database, which is incorporated herein by reference in its entirety.

Any site in the Human AAVS1 locus can be chosen as the target site. For example, the target sequence of the AAVS1 locus can be chosen from GenBank: AC010327.8 (7774 . . . 11429).

### CD38

CD38 (cluster of differentiation 38), also known as cyclic ADP ribose hydrolase is a glycoprotein found on the surface of many immune cells (white blood cells), including CD4+, CD8+, B lymphocytes and natural killer cells. CD38 also functions in cell adhesion, signal transduction and calcium signaling (see, e.g., Orciani M, Trubiani O, Guarnieri S, Ferrero E, Di Primio R (October 2008). “CD38 is constitutively expressed in the nucleus of human hematopoietic cells”. Journal of Cellular Biochemistry. 105 (3): 905-12; and “Entrez Gene: CD38 molecule”.). In humans, the CD38 protein is encoded by the CD38 gene which is located on chromosome 4. CD38 is a paralog of CD157, which is also located on chromosome 4 (4p15) in humans. More details regarding CD38 locus can be found in NCBI database, which is incorporated herein by reference in its entirety.

CD38 has 8 exons and 7 introns. Any site in the CD38 locus can be chosen as the target site. For example, the target sequence of the CD38 locus can be chosen from GenBank NM_001775.4. Examples of the target sequence of the CD38 locus may comprise exon1 (1 . . . 320), exon2 (321 . . . 450), exon3 (451 . . . 586), exon4 (587 . . . 672), exon5 (673 . . . 746), exon6 (747 . . . 839), exon7 (840 . . . 926) and exon8 (927 . . . 5620).

### NKG2A

NKG2 also known as CD159 (Cluster of Differentiation 159) is a receptor for natural killer cells (NK cells). There are 7 NKG2 types: A, B, C, D, E, F and H. NKG2A dimerizes with CD94 to make an inhibitory receptor (CD94/NKG2). Human NKG2A is located from position 10442264 to 10454685 in Chromosome 12 according to GRCh38.p14. The Gene ID for human NKG2A is 3821. More details regarding this gene can be found in NCBI database, which is incorporated herein by reference in its entirety.

NKG2A has 9 exons and 8 introns. Any site in the NKG2A locus can be chosen as the target site. For example, the target sequence of the NKG2A locus can be chosen from GenBank NM_001304448.1. Examples of the target sequence of the NKG2A locus may comprise exon1 (1 . . . 354), exon2 (355 . . . 426), exon3 (427 . . . 644), exon4 (645 . . . 740), exon5 (741 . . . 794), exon6 (795 . . . 946), exon7 (947 . . . 1047), exon8 (1048 . . . 1142) and exon9 (1143 . . . 1179).

Various embodiments of the above two aspects as well as other aspects will be detailed together below.

PSCs include embryonic stem cells (ESCs), and/or induced pluripotent stem cells (iPSCs). It is preferable that the PSCs are iPSCs because iPSCs represent unlimited cell source for cell-based therapy. ESCs (e.g., hESCs) and iPSCs (e.g., hiPSCs) are known in the art and can be readily obtained using conventional methods, for example, those described in the existing technologies, or commercially available products. For example, CytoTune iPS 2.0 Sendai Reprogramming Kit (ThermoFisher Scientific) can be used to reliably generate induced pluripotent stem cells (iPSCs) from somatic cells, including PBMCs and T-cells.

The genetically modified PSCs and iNK cells comprise an expression cassette comprising one or more exogenous polynucleotides of interest. Any suitable exogenous polynucleotide can be used in the genetically modified PSCs and iNK cells as described herein. The one or more exogenous polynucleotides of interest include, but are not limited to a polynucleotide encoding luciferase or fluorescent protein, a polynucleotide encoding an Fc receptor, a polynucleotide encoding an antibody, a polynucleotide encoding a cytokine, a polynucleotide encoding a protein having safety switch function, or any combination thereof. As a result, the gene editing strategy described herein can provide an efficient, reliable, and targeted approach for stably generating the genetically-modified cells having the functionality as intended.

In some embodiments, the one or more exogenous polynucleotides of interest comprises polynucleotide encoding luciferase or fluorescent protein. Examples of the luciferase or fluorescent protein include, but are not limited to luciferase, Antares2 and Green fluorescent protein (GFP). In some embodiments, the luciferase or fluorescent protein is luciferase. In some embodiments, the luciferase is Antares2 (SEQ ID NO.: 1). In some embodiments, the luciferase is GFP (SEQ ID NO.: 2). Further, the luciferase or fluorescent protein also comprises proteins encoded by other reporter genes. Examples of the other reporter genes include, but are not limited to Chloramphenicol acetyltransferase (Cat) gene, Luciferase (Luc) gene, β-Glucosidase (Gus) gene, and Secreted alkaline phos-phosphatase (Seap) gene.

In some embodiments, the iNK cells described herein exhibit enhanced antibody-dependent cell-mediated cytotoxicity (ADCC). In certain embodiments, the one or more exogenous polynucleotides of interest comprise a polynucleotide encoding an Fc receptor. In certain embodiments, the Fc receptor comprises CD16, CD64 or variants thereof. In certain embodiments, the Fc receptor comprises a non-cleavable CD16A, or a CD64/16A fusion protein comprising a CD64 extracellular domain, a CD16A transmembrane domain, and a CD16A cytoplasmic domain.

Humans express three classes of FcγRs: FcγRI (CD64), FcγRII (CD32), and FcγRIII (CD16). Human NK cells mediate ADCC exclusively by the Fc gamma receptor CD16A (FcγRIIIA), which binds to IgG1 and IgG3. CD16A associates with FcRγ and/or CD3ζ chains and is a potent activating receptor. CD16A engagement alone can trigger NK cell degranulation, whereas other NK cell activating receptors typically require a combination of signaling events to induce degranulation. CD16A is tightly regulated and this includes its rapid downregulation by a proteolytic process upon NK cell activation. This process is referred to as ectodomain shedding and is primarily mediated by the membrane-associated protease ADAM17 (a disintegrin and metalloproteinase-17). CD16A shedding occurs in a cis manner at a specific extracellular location proximal to the cell membrane (see, e.g., Walcheck et al., Expert Opinion on Biological Therapy, Volume 19, 2019-Issue 12).

CD16A is intrinsically a low affinity FcγR, though two allelic variants of the receptor vary in their binding affinity for IgG. Non-cleavable CD16A refers to a natural or non-natural variant of CD16. An exemplary non-cleavable CD16A is high affinity non-cleavable CD16A with 158V/V mutation (SEQ ID NO.: 3). This non-cleavable CD16A is engineered into iPSCs to create hnCD16-iNK cells. Compared with unmodified iNK cells, hnCD16-iNK cells are highly resistant to activation-induced cleavage of CD16A and exhibit enhanced antibody-dependent cellular cytotoxicity (ADCC).

CD64 is an integral membrane glycoprotein known as an Fc receptor that binds monomeric IgG-type antibodies with high affinity. It is also known as Fc-gamma receptor 1 (FcγRI) or FCRI. After binding IgG, CD64 interacts with an accessory chain known as the common γ chain, triggering cellular activation. Structurally, CD64 is composed of a signal peptide that allows its transport to the surface of a cell, three extracellular immunoglobulin domains of the C2-type used to bind antibody, a hydrophobic transmembrane domain, and a short cytoplasmic tail. CD64 is the only high affinity FcγR family member and binds to the same IgG isotypes as CD16A (IgG1 and IgG3) but with >30-fold higher affinity. CD64 is distinguished from the other FcγR members by its unique third extracellular domain, which contributes to its high affinity and stable binding to soluble monomeric IgG. CD64 (FcγRI) is normally expressed by certain myeloid cells but not by NK cells. An exemplary CD64 is shown in SEQ ID NO.: 4.

CD64/16A is a chimeric CD16 receptor with the ectodomain of CD16A replaced with CD64 ectodomain, which also has high affinity and non-cleavable feature that improves the ADCC function. CD64/16A is preferably engineered into iPSCs to create CD64/16A-iNK cells because the iNK cells expressing CD64/16A fusion protein has higher ADCC than the iNK cells expressing CD16A. An exemplary CD64/16A is shown in SEQ ID NO.: 5.

In some embodiments, the antibody used with the provided iNK cells or the composition comprising the provided iNK cells includes, but are not limited to one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, certuximab, dinutuximab, avelumab, daratumumab, isatuximab, elotuzumab, and their humanized or Fc modified variants or fragments and their functional equivalents and biosimilars. In some embodiments, the antibody used with the provided iNK cells or the composition comprising the provided iNK cells comprises rituximab. In some embodiments, the antibody used with the provided iNK cells or the composition comprising the provided iNK cells comprises trastuzumab. A skilled in the art can readily determine the concentration of this antibody.

In certain embodiments, the one or more exogenous polynucleotide of interest comprises a polynucleotide encoding a cytokine. Cytokines are a class of small molecular proteins with a wide range of biological activities synthesized or secreted by immune cells (such as monocytes, macrophages, T cells, B cells, NK cells, etc.) and certain non-immune cells (endothelial cells, epidermal cells, fibroblasts, etc.). Cytokines generally regulate cell growth, differentiation and effects by binding to corresponding receptors, and regulate immune responses. Cytokines can be divided into interleukins, interferons, tumor necrosis factor superfamily, colony-stimulating factors, chemokines and growth factors, etc. Examples of the cytokine include, but are not limited to ILs such as IL2, IL4, IL6, IL7, IL9, IL10, IL11, IL12, IL15, IL18, or IL21, chemokines such as CXCR2, macrophage inflammatory protein (MIP)-1 alpha, IFN-inducible protein-10 (IP-10), RANTES, monocyte chemotactic protein-1 (MCP-1), MCP-2, and MCP-3, any variant thereof or any combination thereof. In certain embodiments, the cytokine comprises a soluble cytokine. Examples of the soluble cytokine include, but are not limited to NeoIL2 (SEQ ID NO.: 6), IL15RAsu (SEQ ID NO.: 7), IL15RLI (SEQ ID NO.: 8), IL15ILR (SEQ ID NO.: 9) and IL15-TPA (SEQ ID NO.: 10). In certain embodiments, the cytokine comprises a membrane-bound cytokine. As compared with the soluble cytokine, the membrane-bound counterpart is preferable because this cytokine is self-sustaining, which is safer in clinical applications. Preferable examples of the membrane-bound cytokine comprise a membrane-bound IL-15 or variants thereof. More preferable examples of the membrane-bound cytokine comprise mbIL15 (SEQ ID NO.: 11), mbIL15RLI (SEQ ID NO.: 12), and mbIL21-mbIL15 (SEQ ID NO.: 13). A most preferable example of the membrane-bound cytokine comprises mbIL15RLI (SEQ ID NO.: 12). In this strategy, for example, the function of promoting cell proliferation and/or migration can be imparted to the NK cells.

In certain embodiments, the one or more exogenous polynucleotides of interest comprise a polynucleotide encoding an antibody. Examples of the antibody include, but are not limited to, anti-CD19 antibody, anti-CD20 antibody, anti-BCMA antibody, and anti-PDL1 antibody. Examples of the anti-CD19 antibody include, but are not limited to, tafasitamab, loncastuximab and tesirine-lpyl. Examples of the anti-CD20 antibody include, but are not limited to, rituximab, obinutuzumab, and ofatumumab. Examples of the anti-BCMA antibody include, but are not limited to, belantamab and SEA-BCMA. Examples of the anti-PDL1 antibody include, but are not limited to, membrane bound PDL 1 nano-antibody shown in SEQ ID NO.: 79.

In certain embodiments, the one or more exogenous polynucleotides of interest comprise a polynucleotide encoding a protein having safety switch function (also referred to as suicide gene). Examples of the protein having safety switch function, include, but are not limited to, caspase 3, 6, 7 or 9, thymidine kinase, cytosine deaminase, and any combination thereof. In this strategy, a suicide gene is introduced to further improve the safety and controllability of the NK cells.

In certain embodiments, the expression cassette further comprises a linker. The two or more exogenous polynucleotides can be linked to each other by the linker. In some embodiments, the linker encodes a self-cleaving peptide. In some embodiments, the linker include, but are not limited to an Internal Ribosome Entry Sequence (IRES) or 2A self-cleaving peptide. Examples of the 2A self-cleaving peptide include, but are not limited to F2A, E2A, P2A, and T2A.

In the present disclosure, the expression cassette comprises one or more promoters operably linked to the one or more exogenous polynucleotides of interest. The promoter is a part of the gene, usually located upstream of the 5′ end of the structural gene, and is a DNA sequence that RNA polymerase recognizes, binds and initiates transcription. Any suitable promoter can be used in the genetically-modified PSCs and iNK cells described herein. Suitable promoters include, but are not limited to, cytomegalovirus (CMV) promoter. This promoter is a strong constitutive promoter capable of driving high-level expression of any polynucleotide sequence operably linked thereto. Another example of a suitable promoter is elongation growth factor-1α (EF-1α). However, other promoter sequences can also be used, including but not limited to Ubiquitin C (UBC) promoter, Phosphoglycerate Kinase (PGK) promoter, CMV early enhancer/chicken beta actin (CAG) promoter, and CpG free promoter (CLP) promoter. Further, the use of any promoter or variant derived from the above promoters is also contemplated. The present disclosure includes modified nucleotide sequences obtained by the substitution, deletion and/or addition of one or more bases compared with the above promoter sequences, and the modification still retains the biological function of the promoter's high-efficiency expression in activated NK cells. In some embodiments, the present disclosure includes sequences having at least 95%, at least 97% or at least 99% sequence identity to any of the above promoter sequences, and possessing the biological function of highly expressed in activated NK cells.

In preferable embodiments, the one or more promoters are selected from an EF1α promoter, a CMV promoter, and/or CLP promoter. In more preferable embodiments, the one or more promoters are selected from an EF1α promoter, and/or a CMV promoter. In most preferable embodiments, the one or more promoters are selected from an EF1α promoter.

The one or more promoters are operably linked to the one or more exogenous polynucleotides of interest. In certain embodiments, the expression cassette comprises two or more exogenous polynucleotides, and all exogenous polynucleotides can be driven by a common promoter. In certain embodiments, the expression cassette comprises two or more exogenous polynucleotides, and these exogenous polynucleotides can be separately driven by different promoters.

In certain embodiments, the expression cassette further comprises other regulatory sequences for gene expression. Examples of the regulatory sequences include, but are not limited to, enhancer, poly(A) tailing signal sequence, and the like.

Enhancer refers to a DNA sequence that increases the transcription frequency of genes linked to it, and enhancers increase the transcription of downstream genes through the promoter. Effective enhancers can be located at the 5′ end of the gene, or at the 3′ end of the gene, and some can also be located in the intron of the gene. The enhancer can increase the transcription frequency for gene. Examples of the enhancer include, but are not limited to, CMV enhancer, SV40 enhancer, HPV16 LCR enhancer, immunoglobulin heavy chain enhancer, HACNS1 enhancer, GADD45G enhancer, hormone responsive element (HRE), metal-regulated enhancer element (MRE).

In certain embodiments, the expression cassette further comprises a selectable marker gene to screen the expressing cells from a population of cells transfected with the vector. Useful selectable marker gene includes, for example, antibiotic resistance genes such as Kanamycin (Kan), Neomycin (Neo), Tetracycline (Ter), Chloramphenicol (Cam), and the like.

When the expression cassette comprises a selectable marker gene, it can be linked to the the exogenous polynucleotide via a linker. Examples of the linker include, but are not limited to IRES, F2A, E2A, P2A, and T2A.

Further, the continuous or prolonged expansion of the engineered PSCs also affects the gene silencing of the transgene. For example, with the continuous or prolonged expansion of the engineered PSCs, the transgene in the derivative iNK cells also encounter serious gene silencing effect. The present inventors further found that the gene silencing due to the prolonged expansion of the engineered PSCs can be rescued by introducing a Ubiquitous Chromatin Opening Element(s) (UCOE) upstream the promoter(s). Thus, the expression cassette of the present disclosure further comprises one or more Ubiquitous Chromatin Opening Element (UCOE) operably linked to the promoter(s). Based on the above embodiments, the iNK cells can stably maintain the high expression of the one or more exogenous polynucleotides of interest even if the engineered PSCs had been passaged for multiple passages (e.g., at least 10 passages) during the prolonged expansion.

When the expression cassette comprises two or more exogenous polynucleotides separately driven by different promoters, two or more different or same UCOE elements can be operably linked to these promoters, respectively.

Examples of the UCOE include, but are not limited to, 1550F (SEQ ID NO.: 14), 1550R (SEQ ID NO.: 15), 1194F (SEQ ID NO.: 16), 1194R (SEQ ID NO.: 17), 458F (SEQ ID NO.: 18), 458R (SEQ ID NO.: 19), 396F (SEQ ID NO.: 20), 396R (SEQ ID NO.: 21), and SRF6-3F (SEQ ID NO.: 22). In certain preferable embodiments, the UCOE comprises 1550F, 1550R and SRF6-3F. In certain more preferable embodiments, the UCOE comprises 1550F.

In some embodiments, the expression cassette further includes insulators flanking the exogenous polynucleotides of interest. An insulator is a type of cis-regulatory element known as a long-range regulatory element. Examples of the insulators include, but are not limited to, cHS4 (2 copies, SEQ ID NO.: 23).

In certain embodiments, the expression cassette comprises the combination of 1550F with EF1a, or the combination of 1550F with CMV. In certain embodiments, the expression cassette comprises the combination of 1550F with EF1a. As compared with the combination of 1550F with CMV, the combination of 1550F with EF1α is more preferable because this combination can improve the persistency of anti-silencing effect during derivation of iNK cells from the expanded engineered PSCs. In other words, the combination of 1550F with EF1α can block the gene silencing in both iNK differentiation and subsequent iNK expansion and/or maturation, whereas the combination of 1550F with CMV can only block the gene silencing in iNK differentiation.

In certain embodiments, the genetically-modified iNK cells are immature genetically-modified iNK cells. In certain embodiments, the genetically-modified iNK cells are mature genetically-modified iNK cells. These immature and mature genetically-modified iNK cells, and in particular, the mature genetically-modified iNK cells, can still have high expression level and expression uniformity for the one or more exogenous polynucleotides of interest.

In certain embodiments, the genetically-modified iNK cells can have substantially sustained high expression level and expression uniformity for the one or more exogenous polynucleotides of interest even if the genetically-modified PSCs have been passaged for multiple passages during the prolonged expansion. In certain embodiments, the genetically-modified iNK cells can have substantially sustained high expression level and expression uniformity for the one or more exogenous polynucleotides of interest even if the genetically-modified PSCs have been passaged for at least 3, 4 or 5 passages during the prolonged expansion. In certain embodiments, the genetically-modified iNK cells can have substantially sustained high expression level and expression uniformity for the one or more exogenous polynucleotides of interest even if the genetically-modified PSCs have been passaged for at least 6, 7, 8, or 9 passages during the prolonged expansion. In certain embodiments, the genetically-modified iNK cells can have substantially sustained high expression level and expression uniformity for the one or more exogenous polynucleotides of interest even if the genetically-modified PSCs have been passaged for at least 10, 11, 12 or 13 passages during the prolonged expansion. In certain embodiments, the genetically-modified iNK cells can have substantially sustained high expression level and expression uniformity for the one or more exogenous polynucleotides of interest even if the genetically-modified PSCs have been passaged for at least 14, 15, 16, 17, 18, 19, or 20 passages during the prolonged expansion.

### Methods for Producing Genetically Modified Cells

The present disclosure also relates to methods and compositions for producing the genetically-modified PSCs and iNK cells described herein.

In further aspect, provided herein is a method for producing genetically-modified pluripotent stem cells (PSCs), comprising: introducing into PSCs a construct comprising a site-specific endonuclease capable of introducing a double strand break at a selected locus of the genome of the PSCs and a construct comprising an expression cassette comprising one or more exogenous polynucleotides of interest and one or more promoters operably linked to the one or more exogenous polynucleotides of interest and a pair of homology arms specific to the selected locus and flanking the expression cassette; and integrating the expression cassette comprising the one or more exogenous polynucleotides of interest and the one or more promoters into the genome of the PSCs at the selected locus via homologous recombination by the endonuclease to obtain the genetically-modified PSCs, wherein the selected locus is CISH and/or Rosa26; wherein the genetically-modified PSCs are capable of differentiation into genetically-modified iNK cells having higher expression level and uniformity for the one or more exogenous polynucleotides of interest as compared with the PSCs genetically modified under the same conditions except for the integration at a locus other than CISH and/or Rosa26.

According to the present method, the genetically-modified PSCs are capable of differentiation into the iNK cells at the efficiency of at least 90%, such as for example, 91% or more, 92% or more, 93% or more, 94% or more, or 95% or more. In certain embodiments, the genetically-modified PSCs are capable of differentiation into the iNK cells at the efficiency of at least 96%. In certain embodiments, the genetically-modified PSCs are capable of differentiation into the iNK cells at the efficiency of at least 97%. In certain embodiments, the genetically-modified PSCs are capable of differentiation into the iNK cells at the efficiency of at least 98%. In certain embodiments, the genetically-modified PSCs are capable of differentiation into the iNK cells at the efficiency of at least 99%. In certain embodiments, the genetically-modified PSCs are capable of differentiation into the iNK cells at the efficiency of 100%.

According to the present method, the genetically-modified PSCs are capable of differentiation into genetically-modified iNK cells having at least 5 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 6, 7, 8, 9 or 10 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 11, 12, 13, 14 or 15 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 16, 17, 18, 19 or 20 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 21, 22, 23, 24 or 25 times higher expression level for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least 26, 27, 28, 29 or 30 times higher expression level for the one or more exogenous polynucleotides of interest than the PSCs genetically modified under the same conditions except for the integration at the locus other than CISH and/or Rosa26.

According to the present method, the genetically-modified PSCs are capable of differentiation into genetically-modified iNK cells having at least one time higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least two times higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least three times higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least four times higher expression uniformity for the one or more exogenous polynucleotides of interest than the PSCs genetically modified under the same conditions except for the integration at the locus other than CISH and/or Rosa26. In certain embodiments, the genetically-modified PSCs are capable of differentiation into genetically-modified iNK cells having at least five times higher expression uniformity for the one or more exogenous polynucleotides of interest than the PSCs genetically modified under the same conditions except for the integration at the locus other than CISH and/or Rosa26.

According to the present method, the genetically-modified PSCs are capable of differentiation into iNK cells having at least 90%, such as for example, 91% or more, 92% or more, 93% or more, 94% or more, or 95% or more expression uniformity for the one or more exogenous polynucleotides of interest. In certain embodiments, the genetically-modified PSCs are capable of differentiation into iNK cells having at least 96% expression uniformity for the one or more exogenous polynucleotides of interest. In certain embodiments, the genetically-modified PSCs are capable of differentiation into iNK cells having at least 97% expression uniformity for the one or more exogenous polynucleotides of interest. In certain embodiments, the genetically-modified PSCs are capable of differentiation into iNK cells having at least 98% expression uniformity for the one or more exogenous polynucleotides of interest. In certain embodiments, the genetically-modified PSCs are capable of differentiation into iNK cells having at least 99% expression uniformity for the one or more exogenous polynucleotides of interest. In certain embodiments, the genetically-modified PSCs are capable of differentiation into iNK cells having 100% expression uniformity for the one or more exogenous polynucleotides of interest.

In some embodiments, the selected locus is CISH. In some embodiments, the locus other than CISH and/or Rosa26 is selected from AAVS1, CD38 and/or NKG2A. The gene loci, CISH, Rosa26, AAVS1, CD38 and NKG2A, have been described elsewhere herein (e.g., as described in the Genetically Modified Cells herein), and these same descriptions are omitted herein for purpose of simplification.

Any PSCs can be used in the present method. PSCs include embryonic stem cells (ESCs), and/or induced pluripotent stem cells (iPSCs). It is preferable that the PSCs are iPSCs because iPSCs represent unlimited cell source for cell-based therapy. ESCs (e.g., hESCs) and iPSCs (e.g., hiPSCs) are known in the art and can be readily obtained using conventional methods, for example, those described in the existing technologies, or commercially available products. For example, CytoTune iPS 2.0 Sendai Reprogramming Kit (ThermoFisher Scientific) can be used to reliably generate induced pluripotent stem cells (iPSCs) from somatic cells, including PBMCs and T-cells.

In the present disclosure, the method further comprise expanding the PSCs before the gene modification. Expanding the PSCs before the gene modification comprises passaging the PSCs during the expansion. The technology for expanding and passaging the PSCs are conventional in the art. For example, the PSCs can be cultured and expanded in E8 medium.

In the gene engineering, a recombination vector is generally used to deliver a target gene into cells. In the present disclosure, a donor vector is used to introduce the construct comprising an expression cassette comprising the exogenous polynucleotides of interest and a vector for editing tool is used to introduce the construct comprising a site-specific endonuclease capable of introducing the double strand break (DSB). In certain embodiments, the constructs are vectors. Vectors as used herein generally include, but are not limited to, plasmids, bacteriophages, animal viruses, and cosmids. The vector may be an expression vector, including transient expression vectors, and viral expression vectors. The vector is preferably an eukaryotic expression vector. Viruses that can be used as vectors include, but are not limited to, retroviruses, adenoviruses, adeno-associated viruses, herpes viruses, and lentiviruses. The technology for constructing a recombination vector is common for a skilled artisan in the art of gene engineering.

As a tool for targeted integration described herein, available endonuclease capable of introducing a DSB include, but not limited to, zinc-finger nuclease (ZFN), transcription activator-like effector nuclease (TALEN), and CRISPR-Cas nuclease.

In some embodiments, the endonuclease capable of introducing a double strand break comprises ZFN. As known for a skilled in the art, ZFN is a targeted endonuclease having a nuclease fused to a zinc finger DNA binding domain. A zinc finger is a domain of about 30 amino acids within the zinc finger binding domain whose structure is stabilized through coordination of a zinc ion. Examples of zinc fingers include, but not limited to, C2H2zinc fingers, C3H zinc fingers, and C4zinc fingers. An example of a ZFN is a fusion polypeptide of the FokI nuclease domain with a zinc finger DNA binding domain.

In some embodiments, the endonuclease capable of introducing a double strand break comprises TALEN. TALEN is a targeted endonuclease having a nuclease fused to a TAL effector DNA binding domain. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). An example of a TALEN is a fusion polypeptide of the FokI nuclease domain with a TAL effector DNA binding domain.

In some embodiments, the endonuclease capable of introducing a double strand break comprises CRISPR-Cas nuclease. CRISPR/Cas system is a powerful technology used as gene editing tool to selectively modify DNA sequence at any specific location in the genome of a cell. The CRISPR-Cas systems have been categorized into two classes and six major types. An example of CRISPR/Cas system is CRISPR/Cas9 system. CRISPR-Cas9 system is based on nucleolytic activity of the endonuclease protein, Cas9, which is guided to the desired site in the genome by a specificity determinant RNA, termed as guide RNA (gRNA). Apart from these, another sequence known as protospacer adjacent motif (PAM), present adjacent to the target site, is recognized by the CRISPR/Cas9 system and is crucial for the functionality of Cas9. The Cas9 protein binds to the target location in the presence of gRNA, with high precision and performs a double strand break at the cleavage site. Using predesigned repair template, the knock-in of gene as intended can be achieved by Homology-directed Repair (HDR).

Table 1 lists examples of gRNA targeting sequences for the CISH locus.

TABLE 1

| Exon # | Targeting Sequence | SEQ ID NO | PAM |
|---|---|---|---|
| 2 | TTCTAGACCTCGTCCTTTGC | 24 | TGG |
| 3 | GCACAGGTGTTGCAGGCTGC | 25 | GGG |
| 3 | GGGTTCCATTACGGCCAGCG | 26 | AGG |
| 3 | GTGGAGGAGCAGGCAGTGCT | 27 | GGG |
| 2 | GCCAAAGGTGCTGGACCCAG | 28 | AGG |
| 1 | ACATGGTCCTCTGCGTTCAG | 29 | GGG |

Table 2 lists examples of gRNA targeting sequences on the ROSA26 locus.

TABLE 2

| Target Gene Symbol | Exon # | Targeting Sequence | SEQ ID NO | PAM |
|---|---|---|---|---|
| THUMPD3 | 4 | GTTTAGAGTCACATGCAACA | 30 | GGG |
| THUMPD3 | 3 | TGATCTCAGACAATGAACCT | 31 | GGG |
| THUMPD3 | 5 | TTCTTACCTGAGCATCCCAT | 32 | AGG |
| THUMPD3 | 6 | GGATCGACTATTATATCATA | 33 | AGG |
| THUMPD3 | 2 | ATTGGAGCCACTGTACCTAC | 34 | TGG |
| N/A | N/A | GGCGATGACGAGATCACGCG | 35 | AGG |

Table 3 lists examples of gRNA targeting sequences for AAVS1.

TABLE 3

| Target Gene Symbol | Exon # | Targeting Sequence | SEQ ID NO | PAM |
|---|---|---|---|---|
| PPP1R12C | 2 | TGCCTCACCTGGCGATATCT | 36 | AGG |
| PPP1R12C | 1 | GTGCAGGGCGCTGATACCGT | 37 | CGG |
| PPP1R12C | 3 | GTCCCTAGTGGCCCCACTGT | 38 | GGG |
| PPP1R12C | 4 | GCCAGCACCTCGTGTCATGA | 39 | AGG |
| PPP1R12C | 5 | GGCACTGGCCCTCACCGCAT | 40 | GGG |
| PPP1R12C | 6 | CAGGCTCAGTACTTCCTCAT | 41 | CGG |

Table 4 lists examples of gRNA targeting sequences for CD38 locus.

TABLE 4

| Exon # | Targeting Sequence | SEQ ID NO | PAM |
|---|---|---|---|
| 3 | TCTGGCCCATCAGTTCACAC | 42 | AGG |
| 2 | TGAGTTCCCAACTTCATTAG | 43 | TGG |
| 1 | GGCCAACTGCGAGTTCAGCC | 44 | CGG |
| 5 | CATCACATGGACCACATCAC | 45 | AGG |
| 4 | CTGGAAAACGGTTTCCCGCA | 46 | GGG |
| 8 | GATGTGCAAGATGAATCCTC | 47 | AGG |

Table 5 lists examples of gRNA targeting sequences for NKG2A locus.

TABLE 5

| Exon # | Targeting Sequence | SEQ ID NO | PAM |
|---|---|---|---|
| 2 | GGTCTGAGTAGATTACTCCT | 48 | TGG |
| 7 | TATTATTGAAGATCCACACT | 49 | GGG |
| 5 | TCCAACAGTTGTTACTACAT | 50 | TGG |
| 3 | GCTCCAGAGAAGCTCATTGT | 51 | TGG |
| 4 | TTCTAGCTACATTAATACAG | 52 | AGG |
| 6 | ATGGGTGACAATGAATGGTT | 53 | TGG |

A target gene can be knocked-in via homologous recombination. Homologous recombination is a form of genetic recombination in which two similar DNA strands exchange genetic material. It is also called DNA crossover. Homologous recombination occurs between two homologous DNA molecules. The technologies for designing and acquiring sequences of homologous arms are generally common for a skilled artisan in the art. Based on the principle of homologous recombination, a skilled artisan can readily determine the sequences of homologous arms according to the selected target site for gene editing.

Similarly to the vector for editing tool, a donor vector is constructed to introduce the construct comprising an expression cassette comprising the exogenous polynucleotides of interest.

In the present method, any suitable exogenous polynucleotide can be used as intended. The one or more exogenous polynucleotides of interest include, but are not limited to a polynucleotide encoding luciferase or fluorescent protein, a polynucleotide encoding an Fc receptor, a polynucleotide encoding an antibody, a polynucleotide encoding a cytokine, a polynucleotide encoding a protein having safety switch function, or any combination thereof.

In some embodiments, the one or more exogenous polynucleotides of interest comprise a polynucleotide encoding luciferase or fluorescent protein. In some embodiments, the luciferase or fluorescent protein also comprises proteins encoded by other reporter genes. Examples of the luciferase or fluorescent protein and the other reporter genes have been described elsewhere herein (e.g., as described in the Genetically Modified Cells herein), and these same descriptions are omitted herein for purpose of simplification. In some embodiments, the luciferase is Antares2 (SEQ ID NO.: 1). In some embodiments, the luciferase is GFP (SEQ ID NO.: 2).

In certain embodiments, the one or more exogenous polynucleotides of interest comprise a polynucleotide encoding an Fc receptor. In certain embodiments, the Fc receptor comprises CD16, CD64 or variants thereof. In certain embodiments, the Fc receptor comprises a non-cleavable CD16A, or a CD64/16A fusion protein comprising a CD64 extracellular domain, a CD16A transmembrane domain, and a CD16A cytoplasmic domain.

CD16, CD64 and CD64/16A fusion protein have been described elsewhere herein (e.g., as described in the Genetically Modified Cells herein), and these same descriptions are omitted herein for purpose of simplification. An exemplary CD16A is non-cleavable CD16A, and in particular a high affinity non-cleavable CD16A with 158V/V mutation (SEQ ID NO.: 3). This non-cleavable CD16A is engineered into iPSCs to create hnCD16-iNK cells. Compared with unmodified iNK cells, hnCD16-iNK cells are highly resistant to activation-induced cleavage of CD16A and exhibit enhanced antibody-dependent cellular cytotoxicity (ADCC). Further, CD64/16A is preferably engineered into iPSCs to create CD64/16A-iNK cells because the iNK cells expressing CD64/16A fusion protein has higher ADCC than the iNK cells expressing CD16A. An exemplary CD64/16A is shown in SEQ ID NO.: 5.

In certain embodiments, the one or more exogenous polynucleotide of interest comprises a polynucleotide encoding a cytokine. The cytokine have been described elsewhere herein (e.g., as described in the Genetically Modified Cells herein), and these same descriptions are omitted herein for purpose of simplification. In certain embodiments, the cytokine comprises a membrane-bound cytokine. In certain embodiments, the cytokine comprises a membrane-bound IL-15 and a variant thereof. In certain embodiments, the cytokine comprises membrane-bound IL15RLI shown in SEQ ID NO.: 12.

In certain embodiments, the one or more exogenous polynucleotides of interest comprise a polynucleotide encoding an antibody. The antibody have been described elsewhere herein (e.g., as described in the Genetically Modified Cells herein), and these same descriptions are omitted herein for purpose of simplification.

In certain embodiments, the one or more exogenous polynucleotides of interest comprise a polynucleotide encoding a protein having safety switch function. The polynucleotide encoding a protein having safety switch function have been described elsewhere herein (e.g., as described in the Genetically Modified Cells herein), and these same descriptions are omitted herein for purpose of simplification.

In certain embodiments, the expression cassette further comprises a linker. The two or more exogenous polynucleotides can be linked to each other by the linker. The linker has been described elsewhere herein (e.g., as described in the Genetically Modified Cells herein), and these same descriptions are omitted herein for purpose of simplification.

In the present method, the expression cassette comprises one or more promoters. The promoters have been described elsewhere herein (e.g., as described in the Genetically Modified Cells herein), and these same descriptions are omitted herein for purpose of simplification. In certain embodiments, the one or more promoters are selected from an exogenous promoter. When an exogenous promoter is used, the one or more promoters are directly operatively linked to the one or more exogenous polynucleotides of interest. In certain embodiments, the one or more promoters are selected from an endogenous promoter comprised in the selected locus. When an endogenous promoter is used, the one or more promoters are operatively linked to the one or more exogenous polynucleotides of interest upon the integration.

In preferable embodiments, the one or more promoters are selected from an EF1α promoter, a CMV promoter, and/or CLP promoter. In more preferable embodiments, the one or more promoters are selected from an EF1α promoter, and/or a CMV promoter. In most preferable embodiments, the one or more promoters are selected from an EF1α promoter.

The one or more promoters are operably linked to the one or more exogenous polynucleotides of interest. In certain embodiments, the expression cassette comprises two or more exogenous polynucleotides, and all exogenous polynucleotides can be driven by a common promoter. In certain embodiments, the expression cassette comprises two or more exogenous polynucleotides, and these exogenous polynucleotides can be separately driven by different promoters.

In certain embodiments, the expression cassette further comprises other regulatory sequences. The other regulatory sequences have been described elsewhere herein (e.g., as described in the Genetically Modified Cells herein), and these same descriptions are omitted herein for purpose of simplification.

The expression cassette may further comprise a selectable marker gene, and a linker for linking the selectable marker gene to the the exogenous polynucleotide. The selectable marker gene and the linker have been described elsewhere herein (e.g., as described in the Genetically Modified Cells herein), and these same descriptions are omitted herein for purpose of simplification.

The expression cassette further comprises one or more Ubiquitous Chromatin Opening Elements (UCOE) operably linked to the one or more promoters. Based on the above embodiments, the iNK cells can stably maintain the high expression of the one or more exogenous polynucleotides of interest even if the engineered PSCs had been passaged for multiple passages (e.g., at least 10 passages) during the prolonged expansion. The UCOE element has been described elsewhere herein (e.g., as described in the Genetically Modified Cells herein), and these same descriptions are omitted herein for purpose of simplification. When the expression cassette comprises two or more exogenous polynucleotides separately driven by different promoters, two or more different or same UCOE elements can be operably linked to these promoters, respectively.

In certain embodiments, the expression cassette further comprises insulators. The insulators have been described elsewhere herein (e.g., as described in the Genetically Modified Cells herein), and these same descriptions are omitted herein for purpose of simplification.

In certain embodiments, the expression cassette comprises the combination of 1550F with EF1a, or the combination of 1550F with CMV. In certain embodiments, the expression cassette comprises the combination of 1550F with EF1a. As compared with the combination of 1550F with CMV, the combination of 1550F with EF1α is more preferable because this combination can improve the persistency of anti-silencing effect during derivation of iNK cells from the expanded engineered PSCs.

The present disclosure also includes a nucleic acid construct comprising an expression cassette comprising one or more exogenous polynucleotides of interest, and one or more promoters operably linked to the one or more exogenous polynucleotides of interest. The nucleic acid construct may further comprise one or more UCOE elements operably linked to the one or more promoters. The exogenous polynucleotides of interest, the promoters and other elements comprising the UCOE element have been described above.

After targeted editing the PSCs, the genetically-modified PSCs can be expanded to provide a unlimited cell source for the production of engineered iNK cells as intended. Therefore, the method of the present disclosure further comprises expanding (e.g., continuously expanding) the genetically-modified PSCs. Any common method for expanding or continuously expanding PSCs can be used in the method herein. For example, the genetically-modified PSCs can be cultured and expanded in E8 medium.

In certain embodiments, continuously expanding the genetically-modified PSCs comprises passaging the genetically-modified PSCs for multiple passages during the expansion. In certain embodiments, continuously expanding the genetically-modified PSCs comprises passaging the genetically-modified PSCs for at least 3, 4 or 5 passages during the expansion. In certain embodiments, continuously expanding the genetically-modified PSCs comprises passaging the genetically-modified PSCs for at least 6, 7, 8, or 9 passages during the expansion. In certain embodiments, continuously expanding the genetically-modified PSCs comprises passaging the genetically-modified PSCs for at least 10, 11, 12 or 13 passages during the expansion. In certain embodiments, continuously expanding the genetically-modified PSCs comprises passaging the genetically-modified PSCs for at least 14, 15, 16, 17, 18, 19, or 20 passages during the expansion.

Also provided herein is a method for producing genetically-modified iNK cells, comprising producing genetically-modified PSCs according to the method for producing genetically-modified PSCs described herein, and then differentiating the genetically-modified PSCs into NK cells, thereby producing the genetically-modified iNK cells, wherein the genetically-modified iNK cells have higher expression level and uniformity for the one or more exogenous polynucleotides of interest as compared with the iNK cells genetically modified under the same conditions except for the integration at a locus other than CISH and/or Rosa26.

As the method for differentiating genetically-modified PSCs into iNK cells, any method of differentiating PSCs into NK cells known in the art can be used. For example, a method can be used, comprising forming a plurality of embryoid bodies from PSCs; and adding a first differentiation medium, wherein the first differentiation medium includes GSK3B inhibitor (e.g., CHIR99021), and at least one of BMP signaling pathway activator (e.g., BMP4), insulin, IGF-1, VEGF, and bFGF in the basal medium; removing the first differentiation medium, and adding the second differentiation medium, wherein the second differentiation medium includes VEGF, bFGF, and at least one of the following components in the basal medium: BMP signaling pathway activator (e.g., BMP4), Nodal inhibitor (e.g., SB431542), insulin, IGF-1, IL3 and IL6; removing the second differentiation medium, and adding the third differentiation medium to obtain hematopoietic progenitor cells, wherein the third differentiation medium includes a growth factor and a colony-stimulating factor in the basal medium, wherein the growth factor is selected from one or more of insulin, VEGF, IGF-1 and bFGF; the colony stimulating factor is selected from one or more of TPO, SCF and Flt-3L; and, removing the third differentiation medium, and adding the fourth differentiation medium to differentiate hematopoietic progenitor cells into NK cells, wherein the fourth differentiation medium includes one or more colony-stimulating factor and one or more interleukin in the basal medium, wherein the colony-stimulating factor is selected from TPO, SCF, Flt-3L, and IL-3, and interleukin is selected from IL-2, IL-7, and IL-15. The method for for differentiating PSCs into NK cells has been described in greater detail in CN111235105B, the entire disclosures of which are incorporated herein by reference.

In certain embodiments, the method for producing genetically-modified iNK cells further comprises expanding and maturing the genetically-modified iNK cells.

As the method for expanding and maturing the genetically-modified iNK cells, any method of expanding and maturing iNK cells known in the art can be used. For example, a method can be used, comprising expanding and maturing NK cells in an expansion and maturation medium comprising interleukin and a substance for promoting NK cell maturation and expansion in the basal medium, wherein the interleukin is selected from one or more of IL-2, IL-12, IL-18, IL-21, IL-27 and IL-15, and the substance for promoting NK cell maturation and expansion is selected from one or more of human AB plasma, human platelet lysate, Vitamin A, nicotinamide, Vitamin E and Heparin, and wherein the basal medium includes IMDM, F-12, 0.2-20 mg/ml rHSA, 0-1000 μM thioglycerol, 0-200 μg/ml ascorbic acid, 1-50 μg/ml transferrin, 1-50 ng/ml sodium selenite, and 5-100 μM ethanolamine. The method of expanding and maturing iNK cells has been described in greater detail in CN111235105B, the entire disclosures of which are incorporated herein by reference.

According to the present disclosure, the production method for genetically-modified iNK cells can provide the genetically-modified iNK cells at a high cell purity or proportion (represented by CD56+ cell percentage or CD56+%). In certain embodiments, the production method for genetically-modified iNK cells can provide the genetically-modified iNK cells at the purity of at least 90%, such as for example, 91% or more, 92% or more, 93% or more, 94% or more, or 95% or more. In certain embodiments, the production method for genetically-modified iNK cells can provide the genetically-modified iNK cells at the purity of at least 96%. In certain embodiments, the production method for genetically-modified iNK cells can provide the genetically-modified iNK cells at the purity of at least 97%. In certain embodiments, the production method for genetically-modified iNK cells can provide the genetically-modified iNK cells at the purity of at least 98%. In certain embodiments, the production method for genetically-modified iNK cells can provide the genetically-modified iNK cells at the purity of at least 99%. In certain embodiments, the production method for genetically-modified iNK cells can provide the genetically-modified iNK cells at the purity of 100%.

The produced genetically-modified iNK cells can be isolated before use. The method for isolation is common in the art, including cell sorting and centrifugation, for example.

### Compositions

The present disclosure further relates to cell populations or compositions comprising the genetically-modified PSCs or iNK cells. In certain embodiments, the present disclosure provides cell populations or compositions comprising the genetically-modified PSCs or iNK cells described herein. In certain embodiments, the present disclosure provides cell populations or compositions comprising the cells produced by any of the production methods described herein.

Also provided herein are pharmaceutical compositions comprising the genetically-modified PSCs or iNK cells described herein, and one or more pharmaceutically acceptable carriers. The amount of cells used in the pharmaceutical composition that is effective in the treatment of a particular disorder or condition can depend on the nature of the disorder or condition and can be determined by standard clinical techniques.

Pharmaceutically acceptable carriers are well known in the art. Exemplary pharmaceutically acceptable carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of cell population used in the pharmaceutical composition that is effective in the treatment of a particular disorder or condition can depend on the nature of the disorder or condition and can be determined by standard clinical techniques.

Methods of formulating suitable pharmaceutical compositions are known in the art (see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Modes of administration include but are not limited to injection and infusion. In certain embodiments, injection includes, without limitation, intravenous, intrathecal, intraperitoneal, intraspinal, intracerebrospinal, and intrasternal infusion. In certain embodiments, the route is intravenous. In certain embodiments, cells described herein are administered as a bolus or by continuous infusion (e.g., intravenous infusion) over a period of time. In certain embodiments, cells described herein are administered in several doses over a period of time (e.g., several infusions over a period of time). The cells described herein can be administered in a single dose or in 2, 3, 4, 5, 6 or more doses (or infusions).

### Use

Also provided herein is use of the genetically-modified PSCs or their derivative cells such iNK cells as disclosed herein in the manufacture of a medicament for treating or preventing tumors such as cancers, autoimmune diseases, infections, or blood diseases.

Also provided herein is a method for treating or preventing cancers, comprising administrating a cell population, a cell composition, or pharmaceutical composition as described herein to a subject in need thereof.

A wide range of cancers can be treated or prevented by administration of a cell population or pharmaceutical composition of the disclosure to a subject in need thereof.

Examples of cancers include, but are not limited to, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, mesothelioma, neuroblastomas, non-Hodgkin's myelomas, nasopharynx cancers, lymphoma, oral cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, chronic myelogenous leukemia, acute: myeloid leukemia, myelomonocytic leukemia, melanoma, large cell membrane lung cancer, ovarian cancer, non-small-cell lung cancer, or small-cell lung cancer, and metastases thereof.

### General Methods

In practicing the present disclosure, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

It should be understood that any aspects or embodiments of the present disclosure described herein, including those described only in the examples or claims, can be combined with any one or more other aspects and/or embodiments of the present disclosure, unless such combination is improper or expressly disclaimed.

### EXAMPLES

### 1. Materials

All common reagents and apparatuses utilized throughout the Examples of the present disclosure are commercially available. The sources of certain reagents and apparatuses have been also described elsewhere herein.

### Example 1

The present example was carried out in order to demonstrate that the Rosa26 and CISH loci significantly outperformed other target loci for engineered hiPSCs and iNK cells.

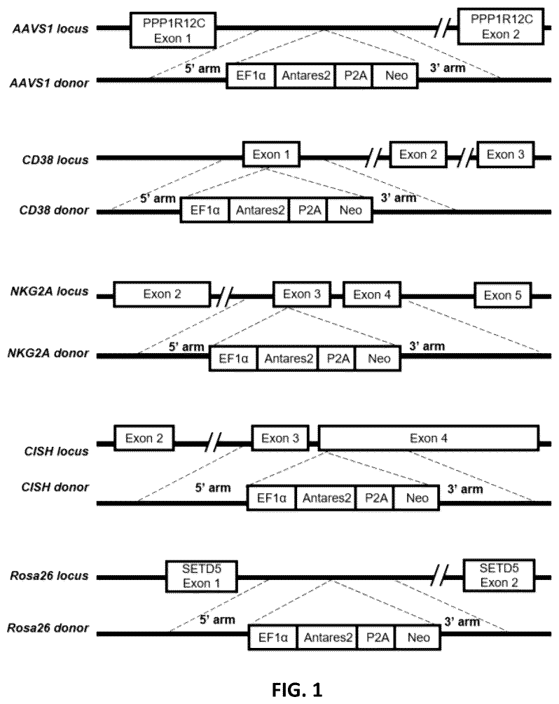

hiPSCs were prepared as per the protocol described in Examples 3 and 4 of the Patent No. CN108373998B. The hiPSCs were cultured and expanded in E8 medium (Nuwacell Co., Ltd., China). The hiPSCs were then engineered at different loci to express Antares2-P2A-Neo as shown in FIG. 1. All procedures were the same except for targeting different loci. The following steps described the details to knock-in Antares2 at a chosen locus (Rosa26, CISH, AAVS1, CD38 or NKG2A).

A donor vector and a Cas/gRNA vector were used to knock-in the target gene Antares2 (SEQ ID NO.: 1) into the chosen locus in the genome of the hiPSCs. In order to construct the Cas/gRNA vector, U6 promoter-gRNA-Locus-gRNA chimeric fragment was synthesized by GenScript Inc. (China) and ligated into KpnI/NotI site of the Cas-Template vector (Nuwacell Co., Ltd.) by T4 ligase (NEB M0202) according to the manufacture's instruction. The constructed Cas/gRNA vector included pUC ori, U6 promoter, gRNA-Locus (e.g., gRNA-CISH), gRNA chimeric fragment (SEQ ID NO.: 65), EF1α promoter, Cas9 gene, P2A linker, Neomycin resistance marker, BGHpA Poly(A) signal and Kanamycin resistance marker. The Cas/gRNA vector and the targeting sequence gRNA-Locus as well as its related location for each locus were shown in Table 6 below.

TABLE 6

| Cas/gRNA Vector | Construct | Chr# | Exon# | Targeting sequence | PAM |
|---|---|---|---|---|---|
| pCas-gRNA-Rosa26 | gRNA-Rosa26 | 3 | N/A | GGCGATGACGAGATCACGCG | AGG |
| | | | | (SEQ ID NO.: 35) | |
| pCas-gRNA-CD38 | gRNA-CD38 | 4 | 1 | GGCCAACTGCGAGTTCAGCC | CGG |
| | | | | (SEQ ID NO.: 44) | |
| pCas-gRNA-NKG2A | gRNA-NKG2A | 12 | 3 | GGTCTGAGTAGATTACTCCT | TGG |
| | | | | (SEQ ID NO.: 48) | |
| pCas-gRNA-CISH | gRNA-CISH | 19 | 4 | GGGTTCCATTACGGCCAGCG | AGG |
| | | | | (SEQ ID NO.: 26) | |
| pCas-gRNA-AAVS1 | gRNA-AAVS1 | 3 | N/A | GTCCCTAGTGGCCCCACTGT | GGG |
| | | | | (SEQ ID NO.: 38) | |

In order to construct the donor vector, a gRNA-5′-terminal homologous arm and a 3′-terminal homologous arm-gRNA were synthesized separately by GenScript Inc. (China) and ligated into NheI/ClaI site and EcoRI/BamHI site of the pKI-Antares2 vector (SEQ ID NO.: 54) (Nuwacell Co., Ltd. China), respectively, by T4 ligase (NEB M0202) according to the manual instruction to obtain a donor vector. The constructed donor vector included pUC ori, 5′-terminal homologous arm, EF1α promoter, Antares2, P2A linker, Neomycin resistance marker, BGHpA PolyA signal, 3′-terminal homologous arm and Kanamycin resistance marker. The homologous arms and the donor vector for each locus were shown in Table 7 below.

TABLE 7

| | 5′-homologous | 3′- homologous | Target |
|---|---|---|---|
| Donor vector | arm | arm | locus |
| pKI-Antares2-Rosa26 | SEQ ID NO.: 55 | SEQ ID NO.: 56 | Rosa26 |
| pKI-Antares2-CISH | SEQ ID NO.: 57 | SEQ ID NO.: 58 | CISH |
| pKI-Antares2-AAVS1 | SEQ ID NO.: 59 | SEQ ID NO.: 60 | AAVS1 |
| pKI-Antares2-CD38 | SEQ ID NO.: 61 | SEQ ID NO.: 62 | CD38 |
| pKI-Antares2-NKG2A | SEQ ID NO.: 63 | SEQ ID NO.: 64 | NKG2A |

2×10⁶ hiPSCs were transfected with 2 μg donor vector and 2 μg Cas/gRNA vector by Nucleofector 2b (Lonza Inc.). The transfected hiPSCs were plated at a cell density of 2×10⁴ cells/cm2 in six well plates and selected with 1 μg/mL puromycin for 1 to 2 days. After growing for 5 to 7 days, hiPSC single clones were picked and transferred into 48 well plates, then further expanded in E8 medium (Nuwacell Co., Ltd.) into 6 well plates to get enough cells for further screening. The hiPSCs from single clones were collected separately and digested into single cells, and the expression of Antares2 was analyzed by flow cytometry assay by recording the fluorescence collected at the PerCP-Cy5.5 tunnel.

Positive clones where the Antares2 sequence was correctly inserted at the chosen locus of the genome were firstly screened by nested PCR assay using the Platinum® Pfx DNA Polymerase (ThermoFisher Scientific) according to the manual instruction. Nested PCR assay was conducted by amplifying the 5′ and 3′ junctions between the inserted sequence and the surrounding genomic target locus of hiPSC genome. Based on the PCR assay, it was confirmed that the clone had both alleles engineered at the desired locus.

For the potential positive clones screened by the PCR assays, sequencing was then conducted by Tsingke Biotechnology Co., Ltd. (China) to further confirm the correct insertion. The positive clones with correct insertion were then karyotyped by KingMed Diagnostics Inc. (China) to exclude chromosomal abnormalities. Finally, the expression cassette of “EF1α-Antares2-P2A-Neo” was successfully inserted at the target site of the chosen locus (as shown in FIG. 1).

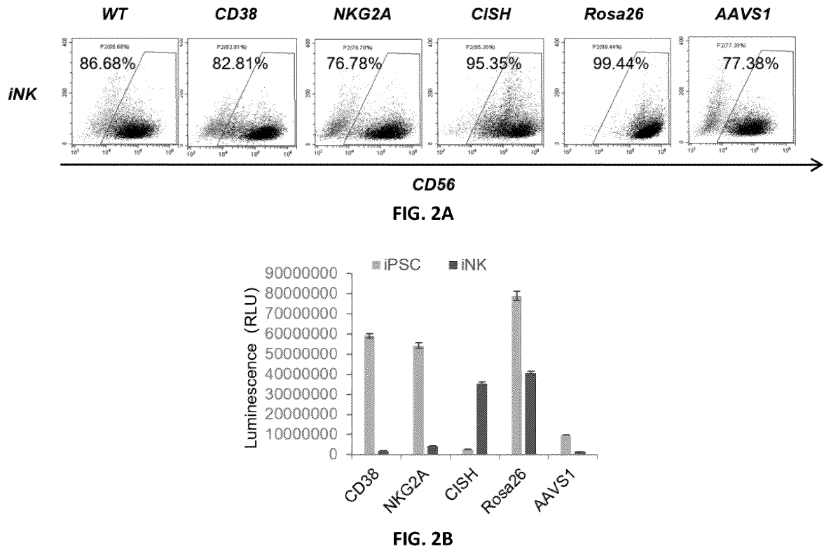

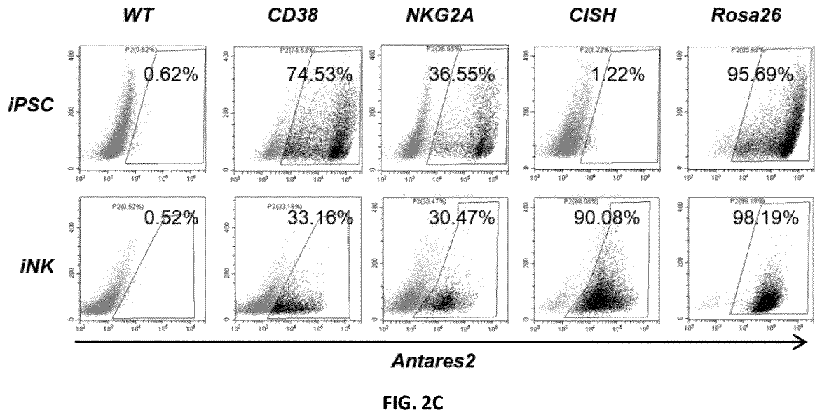

The first passage of confirmed engineered hiPSCs was named as P1 (Passage 1) and the hiPSCs of P1 were then differentiated into iNK cells according to the protocol described in Example 1 of Chinese Patent No. CN111235105B. On day 24 (D24) of differentiation, the iNK cells were collected and stained with anti-CD56-PE antibody (BD, 556647), and the percentage of CD56+ iNK cells was detected by flow cytometry (FIG. 2A).

As shown in FIG. 2A, the hiPSCs with Antares2 integrated at the CISH locus differentiated into iNK cells at the efficiency of 95.35%, and the hiPSCs with Antares2 integrated at the Rosa26 locus differentiated into iNK cells at the efficiency of 99.44%, all of which were similar to 86.68% for wild type (WT) hiPSCs. As controls, the hiPSCs with Antares2 integrated at the locus of AAVS1, CD38 or NKG2A differentiated into iNK cells at the efficiency of 77.38%, 82.81% or 76.78%, respectively. This showed that CISH and Rosa26 did not influence the efficiency of differentiation from hiPSCs to iNK cells.

The expression levels of Antares2 in the engineered hiPSCs and D24 iNK cells were measured with a luciferin-luciferase bioluminescence assay kit (TransDetect® Single-Luciferase (Firefly) Reporter Assay Kit, Transgen (China), Cat. #FR101-01) according to the manual instruction. The results were shown in FIG. 2B. The expressions of Antares2 in the hiPSCs and D24 iNK cells were analyzed by flow cytometry (FIG. 2C).

As shown in FIGS. 2B and 2C, the expression level and expression uniformity of Antares2 integrated at the Rosa26 locus were the highest for both the hiPSCs (95.69% for Antares2⁺ cells) and the INK cells (98.19% for Antares2⁺ cells) among all loci. Among all indicated loci, CISH was unique because the expression of Antares2 integrated at CISH locus was silenced in pluripotent cells but recovered after differentiation into iNK cells (90.08% for Antares2⁺ cells). Except for CISH and Rosa26, all loci had much lower Antares2 expression level and expression uniformity (about 30% for Antares2⁺ cells) after differentiation into iNK cells (lower by about 2 times for uniformity), indicating that the gene expression was silenced or reduced at these loci. This showed that different loci greatly influenced the expression level and activity of the transgene in both pluripotent cells and final differentiated cells.

### Examples 2-3

The present examples were carried out in order to optimize the expression cassettes comprising different forms of cytokines and CD16 for NK cells randomly integrated by PiggyBac transposon. The optimized forms of cytokines and CD16 would be used in engineered hiPSCs and iNK cells.

### Example 2

NK92 cell line was used as an example of NK cells. NK92 cell line was purchased from Procell Inc. (China) and cultured according to the manual instruction. The expression cassettes as shown in Table 8 were synthesized by GenScript Inc. (China) and ligated into XbaI/BamHI site of PB-PNEE vector (SEQ ID NO.: 66) (Nuwacell Co., Ltd. China) separately by T4 ligase (NEB M0202) according to the manual instruction. The CD16A used here was a high affinity non-cleavable CD16 with 158V/V mutation (SEQ ID NO.: 3).

TABLE 8

| No. | Expression Vector | Expression cassette | Sequence |
|---|---|---|---|
| 1 | pPB-PNEE-CD16A-T2A-NeoIL2 | CD16A-T2A-NeoIL2 | (SEQ ID NO.: 67) |
| 2 | pPB-PNEE-CD16A-T2A-IL15RAsu | CD16A-T2A-IL15RAsu | (SEQ ID NO.: 68) |
| 3 | pPB-PNEE-CD16A-T2A-IL15RLI | CD16A-T2A-IL15RLI | (SEQ ID NO.: 69) |
| 4 | pPB-PNEE-CD16A-T2A-IL15ILR | CD16A-T2A-IL15ILR | (SEQ ID NO.: 70) |
| 5 | pPB-PNEE-CD16A-T2A-IL15-TPA | CD16A-T2A-IL15-TPA | (SEQ ID NO.: 71) |
| 6 | pPB-PNEE-CD16A-T2A-mbIL15 | CD16A-T2A-mbIL15 | (SEQ ID NO.: 72) |
| 7 | pPB-PNEE-CD16A-T2A-mbIL15RLI | CD16A-T2A-mbIL15RLI | (SEQ ID NO.: 73) |
| 8 | pPB-PNEE-CD16A-T2A-mbIL21-mbIL15 | CD16A-T2A-mbIL21-mbIL15 | (SEQ ID NO.: 74) |

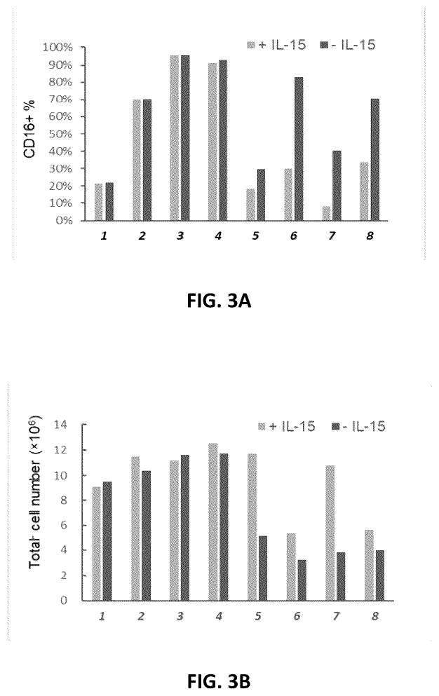

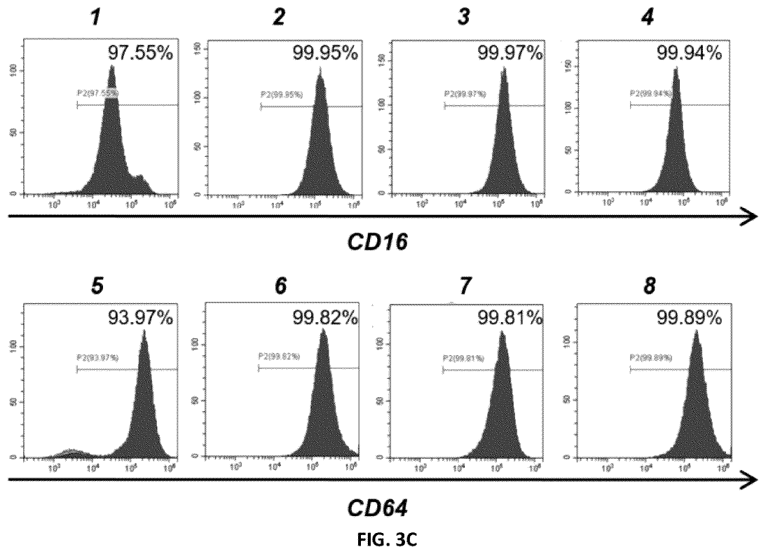

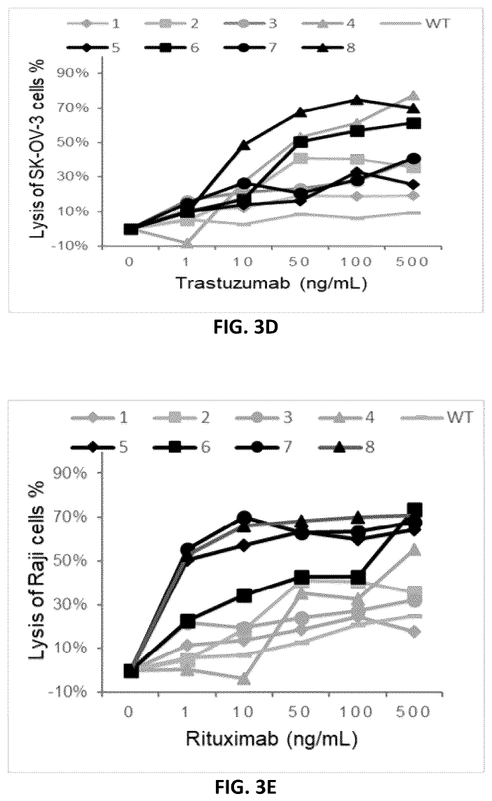

7.5×10⁶ NK92 cells were transfected with 3 μg expression vector and 3 μg PBase vector (Nuwacell Co., Ltd. China). The transfected cells were selected with 750 μg/mL neomycin for about two weeks. NK92 cells stably overexpressing the indicated expression cassette were cultured in NK92 Medium (Procell Inc. China, #CM-0530) in the presence or absence of IL-15 for 7 days. Total cell number was counted on day 7 by Vicell counter (Beckman). The NK92 cells were stained with CD16-APC antibody (APC anti-human CD16 Antibody, Biolegend Cat #302012), the percentage of CD16⁺ cells was analyzed by flow cytometry (FIGS. 3A-3B).

Comparing the percentages of CD16⁺ cells in the presence and absence of IL-15, only NK92 cells expressing membrane-bound IL15 analogues (#6, #7 and #8) had preferential expansion, indicating that the membrane-bound cytokine was self-sustaining which should be safer in clinical applications (FIG. 3A). Among #6, #7 and #8, comparing the total cell numbers in the presence and absence of IL-15, only NK92 cells expressing mbIL15RLI (#7) maintained the best cell proliferation in the presence of IL-15, while #6 and #8 appeared to show inhibitory effect on the proliferation despite the presence of exogenous IL-15 (FIG. 3B).

### Example 3

The expression cassettes shown in Table 9 were synthesized by GenScript Inc. (China) and ligated into XbaI/BamHI site of PB-PNEE vector (Nuwacell Co., Ltd. China) separately by T4 ligase (NEB M0202) according to the manual instruction. The CD16A used here was a high affinity non-cleavable CD16 with 158V/V mutation. CD64/16A used here was a chimeric CD16A receptor with the ectodomain of CD16A replaced with CD64 ectodomain, which also had high affinity and non-cleavable feature.

TABLE 9

| No. | Expression Vector | Expression cassette | Sequence |
|---|---|---|---|
| 1 | pPB-PNEE-CD16A-T2A-NeoIL2 | CD16A-T2A-NeoIL2 | (SEQ ID NO.: 67) |
| 2 | pPB-PNEE-CD16A-T2A-IL15-TPA | CD16A-T2A-IL15-TPA | (SEQ ID NO.: 71) |
| 3 | pPB-PNEE-CD16A-T2A-IL15RLI | CD16A-T2A-IL15RLI | (SEQ ID NO.: 69) |
| 4 | pPB-PNEE-CD16A-T2A-mbIL15RLI | CD16A-T2A-mbIL15RLI | (SEQ ID NO.: 73) |
| 5 | pPB-PNEE-CD64/16A-T2A-NeoIL2 | CD64/16A-T2A-NeoIL2 | (SEQ ID NO.: 75) |
| 6 | pPB-PNEE-CD64/16A-T2A-IL15-TPA | CD64/16A-T2A-IL15-TPA | (SEQ ID NO.: 76) |
| 7 | pPB-PNEE-CD64/16A-T2A-IL15RLI | CD64/16A-T2A-IL15RLI | (SEQ ID NO.: 77) |
| 8 | pPB-PNEE-CD64/16A-T2A-mbIL15RLI | CD64/16A-T2A-mbIL15RLI | (SEQ ID NO.: 78) |

7.5×10⁶ NK92 cells were transfected with 3 μg expression vector and 3 μg PBase vector (Nuwacell Co., Ltd. China). The transfected cells were selected with 750 μg/mL neomycin for about two weeks. The transfected NK92 cells were analyzed for CD16 or CD64 expression by flow cytometry (FIG. 3C). For CD16 expression detection, the randomly integrated NK92 cells were stained with CD16-antibody (APC anti-human CD16 Antibody, Biolegend Cat #302012) and the percentage of CD16⁺ cells was analyzed by flow cytometry. For CD64 expression detection, the randomly integrated NK92 cells were stained with CD64-APC antibody (APC anti-human CD64 Antibody Biolegend Cat #305014) and the percentage of CD64⁺ cells was analyzed by flow cytometry. As shown in FIG. 3C, all randomly integrated NK92 cells had high expression uniformity for the inserted gene.

The ADCC function of the randomly integrated NK92 cells was measured. The ADCC assay was conducted according to the method used in the literature (Dall'Ozzo S, Tartas S, Paintaud G, et al. Rituximab-dependent cytotoxicity by natural killer cells: influence of FCGR3). Specific cytotoxicity of the randomly integrated NK92 cells against SK-OV-3 cells (HER2-expressing ovarian cell line) was measured after a 4-hour incubation of the randomly integrated NK92 cells in the presence of trastuzumab (Roche) with increased concentration (FIG. 3D). Specific cytotoxicity of the randomly integrated NK92 cells against Raji cells (CD20-expressing Burkitt lymphoma cell line) was measured after a 4-hour incubation of the randomly integrated NK92 cells in the presence of Rituximab (Roche) with increased concentration (FIG. 3E). Comparing the ADCC functions of the randomly integrated NK92 cells expressing different forms of CD16, the NK92 cells expressing CD16A significantly outperformed the WT NK92 cells, and the NK92 cells expressing CD64/16A significantly outperformed the NK92 cells expressing 16A (FIGS. 3D and 3E). In particular, the NK92 cells co-expressing CD64/16A and mbIL15RLI (#8) had the best ADCC function.

### Example 4

The present example was carried out in order to demonstrate the effect of different promoter on the transgene expression in the engineered hiPSCs and iNK cells.

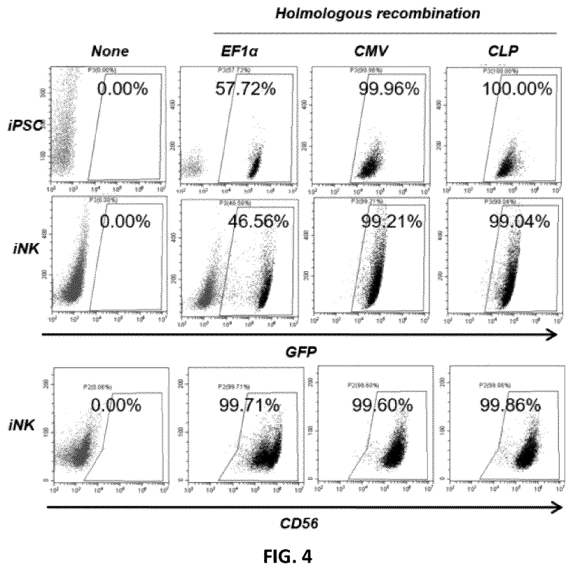

The hiPSCs were cultured and expanded in E8 medium (Nuwacell Co., Ltd., China). GFP driven by EF1a, CMV or CLP promoter was knocked-in at CISH locus in the hiPSCs in similar method to the Example 1. The first passage of confirmed engineered hiPSCs was named as P1 (Passage 1) and the hiPSCs of P1 were then differentiated into iNK cells according to the protocol described in Example 1 of Chinese Patent No. CN111235105B. On Day 31 of differentiation, the iNK cells were collected and stained with PE labelled anti-CD56 antibody (BD, 556647). The GFP expression level and uniformity in the engineered hiPSCs and iNK cells were analyzed by flow cytometry, and the percentage of CD56⁺ NK cells was detected by flow cytometry (FIG. 4).

As shown in FIG. 4, all promoters could support the high expression of inserted GFP during NK cell differentiation, and CMV and CLP promoters outperformed EF1a. In addition, all of the engineered hiPSCs with these promoters could differentiate into NK cells at a high efficiency of 99% or more.

### Example 5

The present example was carried out in order to demonstrate the reduced transgene expression in the iNK cells differentiated from the engineered hiPSCs after prolonged expansion.

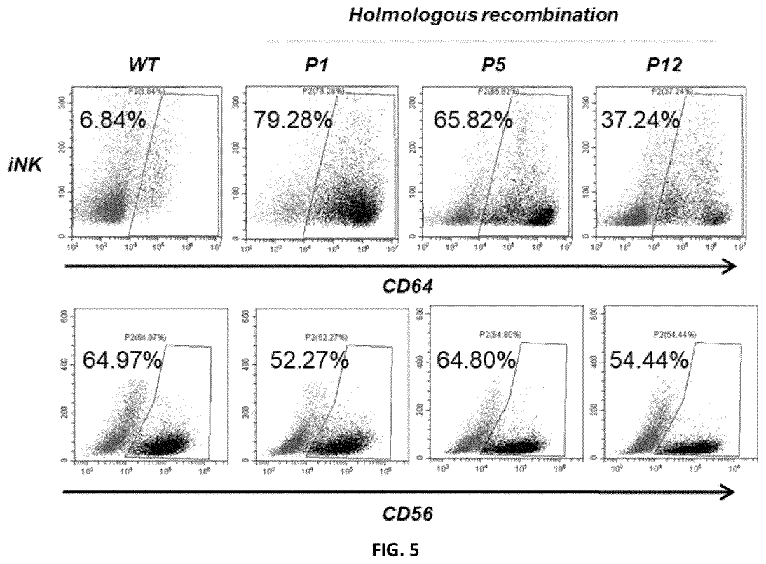

The hiPSCs were cultured and expanded in E8 medium (Nuwacell Co., Ltd., China). CD64/16A-T2A-mbIL15RLI (Example 3, Table 9, #8) driven by EF1α promoter was knocked-in at CISH locus in the hiPSCs with similar method used in the Example 1. The first passage of confirmed CISH^{-/-} CD64/16A knock-in hiPSCs was named as P1 (Passage 1). The hiPSCs of P1 were expanded and passaged in E8 medium (Nuwacell Co., Ltd.) for more than 10 passages. Then, the hiPSCs of P1, P5 and P12 were separately differentiated to iNK cells in similar method to the Example 1. On Day 24 of differentiation, the NK cells were collected and stained with PE labeled anti-CD56 antibody (BD, 556647), the percentage of CD56+NK cells was detected by flow cytometry. The CD64/16A expression and the cell number of the iNK cells were analyzed by flow cytometry (FIG. 5).

As shown in FIG. 5, 79.28% of the iNK cells differentiated from the engineered hiPSCs of P1 expressed CD64/16A, 65.82% of the iNK cells differentiated from the engineered hiPSCs of P5 expressed CD64/16A, and only 37.24% of the iNK cells differentiated from the engineered hiPSCs of P12 expressed CD64/16A, suggesting that the CD64/16A was gradually silenced or had the reduced expression with the prolonged expansion of the engineered hiPSCs. However, the efficiency of differentiation into iNK cells was not significantly influenced with the prolonged expansion of the engineered hiPSCs.

### Examples 6-8

The present examples were carried out in order to demonstrate the effects of various anti-silencing elements on the randomly integrated hiPSC cells.

### Example 6

Different versions of UCOE element (1550F (SEQ ID NO.: 14), 1550R (SEQ ID NO.: 15), 1194F (SEQ ID NO.: 16), 1194R (SEQ ID NO.: 17), 458F (SEQ ID NO.: 18), 458R (SEQ ID NO.: 19), 396F (SEQ ID NO.: 20), and 396R (SEQ ID NO.: 21), with F representing Forward direction and R representing Reverse direction), and mb PDL1 Nb (membrane bound PDL 1 Nano-antibody, shown in SEQ ID NO.: 79) were synthesized by GenScript Inc. (China) and ligated into XbaI/BamHI site and NotI/ClaI site of PB-PNEE vector (Nuwacell Co., Ltd. China) separately by T4 ligase according to the manual instruction (NEB M0202) to obtain recombinant vectors. A Strep tag II was connected to mb PDL1 Nb for easy detection of the Nano-antibody expression.

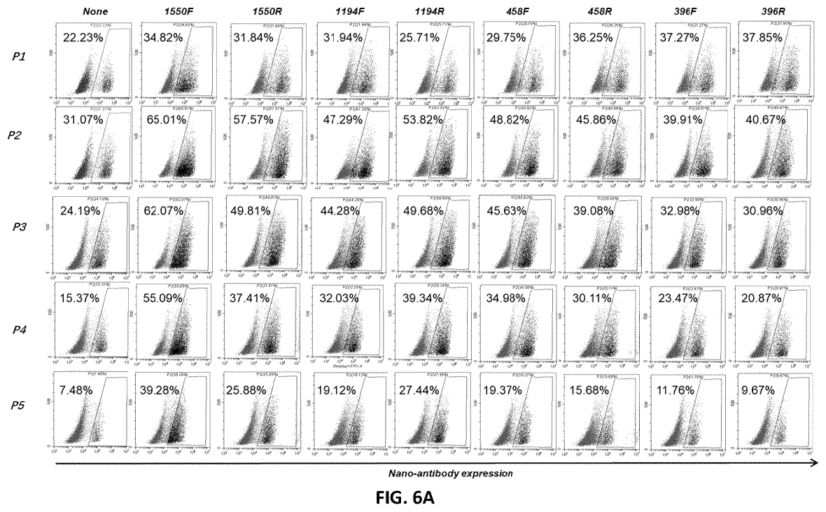

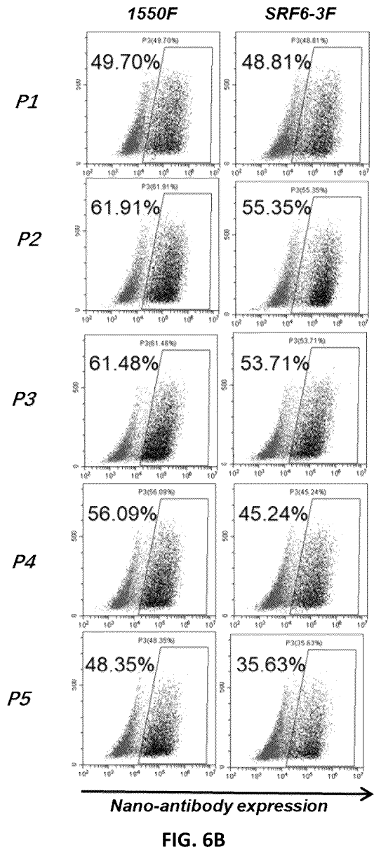

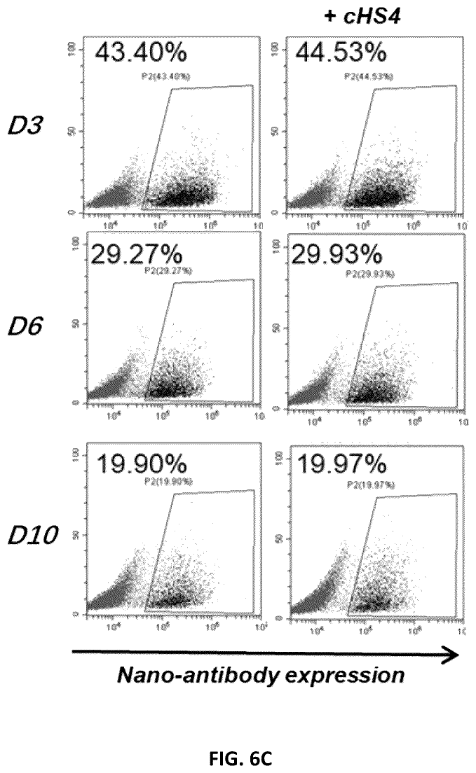

The hiPSC cells were transfected with 3 μg any of the above recombinant vectors and 3 μg PBase vector (Nuwacell Co., Ltd. China). mb PDL1 Nb driven by EF1α promoter with or without different versions of UCOE element was randomly inserted in the hiPSCs by PiggyBac transposon. The hiPSCs stably overexpressing the mb PDL1 Nb were continuously expanded in E8 medium (Nuwacell Co., Ltd.) for 5 passages. On each passage, the cells were collected and stained with Strep tag II antibody (LSBio, Cat #LS-C203631), the percentage of mb PDL1 Nb expressing cells was analyzed by flow cytometry (FIG. 6A).

As shown in FIG. 6A, as compared with the control without any UCOE element, the UCOE element could increase the expression of the Nano-antibody for each passage of the expanded hiPSCs, the length and direction of the UCOE element could influence the anti-silencing effect, and 1550F with the biggest size and forward direction had the best performance in reducing silencing effect. However, while the randomly integrated hiPSCs were continuously expanded and passaged, the gene expression still decreased gradually despite the presence of 1550F, indicating that the UCOE could not completely block the silencing of the Nano-antibody with the prolonged expansion of the randomly integrated hiPSCs.

### Example 7

1550F, SRF6-3F (SEQ ID NO.: 22)), and mb PDL1 Nb were synthesized by GenScript Inc. (China), and mb PDL1 Nb driven by EF1α promoter with 1550F or SRF6-3F was randomly inserted in the hiPSCs in the above similar manner. The hiPSCs overexpressing mb PDL1 Nb were continuously expanded in E8 medium (Nuwacell Co., Ltd.) for 5 passages. On each passage, the cells were collected and analyzed for the expression of mb PDL1 Nb by flow cytometry (FIG. 6B).

In FIG. 6B, comparing SRF6-3F with 1550F, no further improvement in expression stability was noticed.

### Example 8

cHS4 insulator was synthesized by GenScript Inc. (China) and ligated into NotI/ClaI site and HindIII/NdeI site of PB-PNEE vector (Nuwacell Co., Ltd. China) separately by T4 ligase according to the manual instruction (NEB M0202) to obtain a recombinant vector. A Strep tag II was connected to mb PDL1 Nb for easy detection of the Nano-antibody expression.

The hiPSC cells were transfected with 3 μg any of the above recombinant vectors and 3 μg PBase vector (Nuwacell Co., Ltd. China). mb PDL1 Nb driven by EF1α promoter with or without cHS4 insulator was randomly inserted in the hiPSCs by PiggyBac transposon. The stably transfected hiPSC cells were continuously expanded in E8 medium (Nuwacell Co., Ltd.) for 10 days. On Day 3, Day 6 and Day 10, the transfected hiPSCs were collected and analyzed for the expression of mb PDL1 Nb by flow cytometry (FIG. 6C).

As shown in FIG. 6C, comparing the expressions of the Nano-antibody in the presence and the absence of cHS4 insulator, the cHS4 insulator could not rescue the silencing or reduced expression of mb PDL1 Nb with the prolonged expansion of the randomly integrated hiPSCs.

### Example 9

The present example was carried out in order to demonstrate the combined effect of different promoter with a UCOE element in the randomly integrated iNK cells.

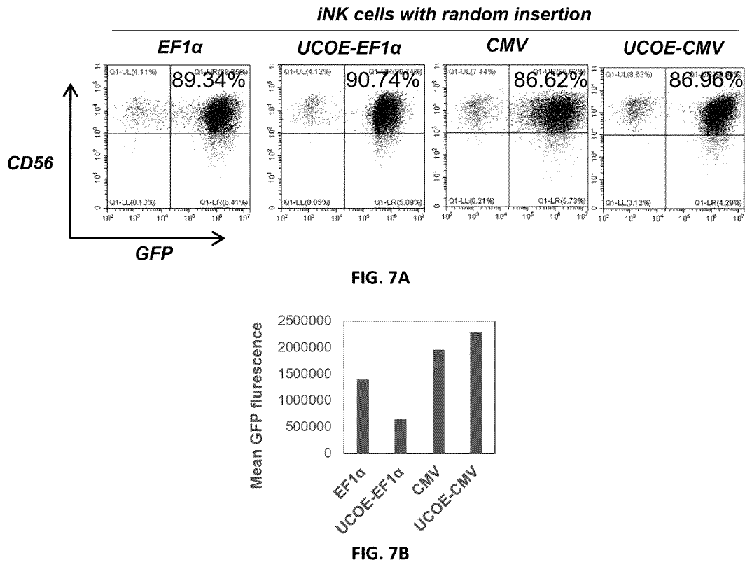

hiPSCs were prepared as per the protocol described in Examples 3 and 4 of the Patent No. CN108373998B, and differentiated into iNK cells according to the protocol described in Example 1 of Chinese Patent No. CN111235105B. GFP driven by different promoter (EF1α and CMV) with or without 1550F UCOE element were randomly inserted in the iNK cells by PiggyBac transposon in similar manner to the Example 2. After expansion (according to the protocol described in Example 1 of Chinese Patent No. CN111235105B) with feeder cells (The Life Ark Inc. China, #ZY-NKZ-0104) and drug selection of 750 μg/mL Neomycin, the randomly integrated iNK cells were collected and analyzed for GFP expression by flow cytometry (FIGS. 7A-7B).

As shown in FIGS. 7A-7B, GFP expression was relatively stable in the iNK cells driven by both CMV promoter and EF1α promoter with or without the UCOE element, indicating that there was no gene silencing when the gene modification was conducted in finally differentiated cell types. CMV promoter could drive higher level of transgene expression with or without UCOE element compared to EF1α promoter. The combination of the UCOE with CMV promoter increased the expression level of the GFP and maintained the expression uniformity thereof while the combination of the UCOE with EF1α promoter decreased the expression level of the GFP and and maintained the expression uniformity thereof. These data indicated that the UCOE-CMV was more effective driver for the transgene expression in the randomly integrated iNK cells.

### Example 10

The present example was carried out in order to demonstrate the anti-silencing effect of a UCOE element on the engineered iNK cells.

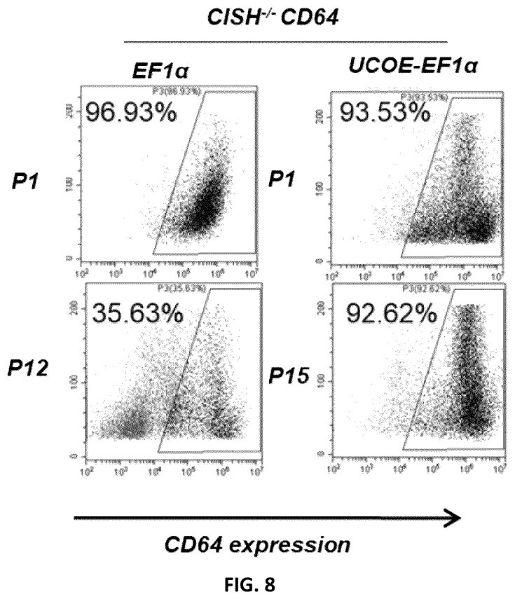

CD64/16A-T2A-mbIL15RLI (Example 3, Table 9, #8) driven by EF1α combined with or without 1550F UCOE element was knocked-in at CISH locus in the hiPSCs in similar method to the Example 1. The first passage of confirmed engineered hiPSCs was named as P1 (Passage 1). The clonal hiPSCs were expanded and passaged in E8 medium (Nuwacell Co., Ltd.) for more than 10 passages. Then, the hiPSCs of P1, P12 and P15 were separately differentiated to iNK cells (P1 and P12 for “EF1α” and P1 and P15 for “UCOE-EF1α”). The CD64 expressions in iNK cells differentiated from the engineered hiPSCs of different passages were analyzed by flow cytometry (FIG. 8).

As shown in FIG. 8, using EF1α promoter without 1550F UCOE element, 96.93% of the iNK cells expressed CD64/16A when they were differentiated from the engineered hiPSCs of P1, while only 35.63% of the iNK cells expressed CD64/16A when they were differentiated from the engineered hiPSCs of P12, indicating that the engineered hiPSCs after the prolonged expansion could give rise to the serious gene silencing or reduced expression. However, using EF1α promoter combined with 1550F UCOE element, the iNK cells could stably maintain the high expression (>90%) of CD64/16A even if the hiPSCs had been expanded and passaged for 15 passages, indicating that the UCOE element could rescue the gene silencing of iNK cells caused by the prolonged expansion of the engineered hiPSCs.

### Example 11

The present example was carried out in order to demonstrate the combined effect of different promoter with a UCOE element on the immature and mature engineered iNK cells.

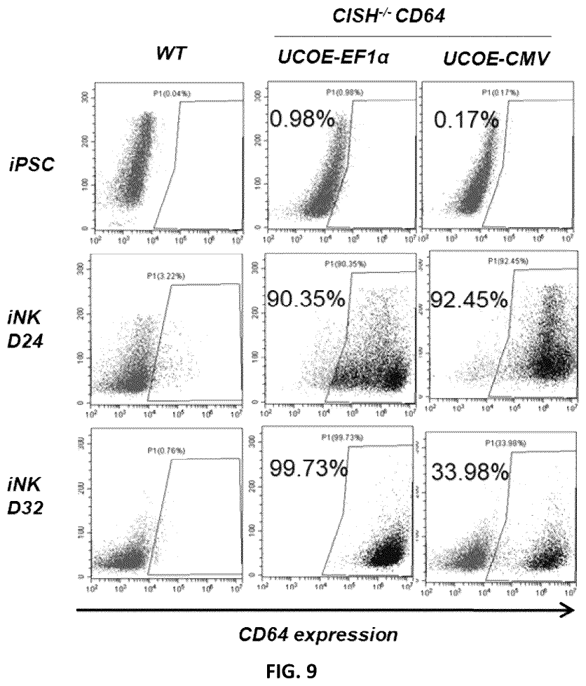

CD64/16A-T2A-mbIL15RLI (Example 3, Table 9, #8) driven by different promoter (EF1α and CMV) combined with 1550F UCOE element was knocked-in at CISH locus in the hiPSCs in similar method to the Example 1. The first passage of confirmed engineered hiPSCs was named as P1 (Passage 1). The hiPSCs of P1 were then differentiated for 24 days to obtain immature iNK cells (D24 iNK cells) according to the protocol described in Example 1 of Chinese Patent No. CN111235105B. The D24 iNK cells were further expanded and matured (according to the protocol described in Chinese Patent No. CN111235105B) for 8 days with feeder cells (The Life Ark Inc. China, #ZY-NKZ-0104) according to the manual instruction to obtain mature iNK cells (D32 iNK cells). The CD64 expressions in the engineered hiPSCs and iNK cells were separately analyzed by flow cytometry on the hiPSCs, D24 iNK cells and D32 iNK cells (FIG. 9).

As shown in FIG. 9, the expression of CD64/16A is silenced in the engineered hiPSCs irrespective of the combination EF1α-1550F or CMV-1550F, and on D24 of iNK differentiation when the cells were still immature, there was no significant difference between the two combinations EF1α-1550F and CMV-1550F in CD64/16A expression. However, when these immature iNK cells were further expanded and matured into mature iNK cells, there was a huge difference between these two combinations, and 1550F combined with EF1α effectively rescued the severe gene silencing in mature iNK cells while 1550F combined with CMV did not.

### Example 12

The present example was carried out in order to demonstrate knock-in of CD16A and mbIL15RLI in the hiPSCs and iNK cells at Rosa26 locus.

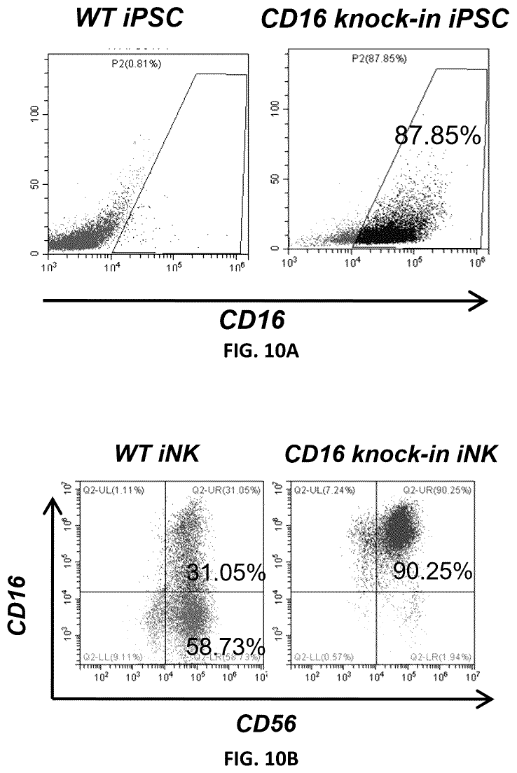

CD16A-T2A-mbIL15RLI (Example 2, Table 8, #7) driven by EF1α promoter was knocked-in at Rosa26 locus in the hiPSCs in similar method to the Example 1. The first passage of confirmed engineered hiPSCs was named as P1 (Passage 1). The hiPSCs of P1 were then differentiated into iNK cells according to the protocol described in Example 1 of Chinese Patent No. CN111235105B. The CD16A expression in the engineered hiPSCs were measured by flow cytometry (FIG. 10A). iNK cells were collected on Day 24 and then co-cultured with feeder cells (The Life Ark Inc. China, #ZY-NKZ-0104) for 9 days according to the manual instruction for further maturation and expansion (according to the protocol described in Chinese Patent No. CN111235105B). The iNK cells on D32 were collected and stained at the same time with anti-CD16 antibody (APC anti-human CD16 Antibody, Biolegend Cat #302012) and anti-CD56 antibody (BD, 556647) for flow cytometry analysis. The results were shown in FIG. 10B.

FIG. 10A showed that the CD64/16A and mbIL15RLI were highly co-expressed in the engineered hiPSCs. FIG. 10B showed that most expanded engineered iNK cells (90.25%) were double positive for both CD16 and CD56, whereas only 31.05% WT iNK cells were double positive, indicating that the gene editing process was safe and allowed high expression of the transgene.

One skilled in the art would readily appreciate that the methods, compositions, and products described herein are representative of exemplary embodiments, and not intended as limitations on the scope of The present disclosure. It will be readily apparent to one skilled in the art that varying substitutions and modifications may be made to the present disclosure disclosed herein without departing from the scope and spirit of The present disclosure.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure is not to be limited in terms of the particular embodiments described in this application, which are intended as single illustrations of individual aspects of the disclosure. All the various embodiments of the present disclosure will not be described herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

1. Genetically-modified pluripotent stem cells (PSCs), comprising an expression cassette integrated at a selected locus of the genome of PSCs, the expression cassette comprising one or more exogenous polynucleotides of interest, and one or more promoters operably linked to the one or more exogenous polynucleotides of interest, wherein the selected locus is CISH and/or Rosa26, and wherein the genetically-modified PSCs are capable of differentiation into genetically-modified iNK cells having higher expression level and uniformity for the one or more exogenous polynucleotides of interest as compared with the PSCs genetically modified under the same conditions except for the integration at a locus other than CISH and/or Rosa26.

2. The genetically-modified PSCs of claim 1, wherein the genetically-modified PSCs are capable of differentiation into genetically-modified iNK cells having at least two times and preferably at least four times higher expression uniformity for the one or more exogenous polynucleotides of interest than the PSCs genetically modified under the same conditions except for the integration at the locus other than CISH and/or Rosa26.

3. The genetically-modified PSCs of claim 1, wherein the locus other than CISH and/or Rosa26 is selected from AAVS1, CD38 and/or NKG2A.

4. The genetically-modified PSCs of claim 1, wherein the one or more promoters are selected from EF1α, PGK, CAG, CMV, CLP, UBC and/or any promoter derived therefrom, and preferably EF1α, CMV, and/or CLP.

5. The genetically-modified PSCs of claim 1, wherein the selected locus is CISH.

6. The genetically-modified PSCs of claim 1, wherein the one or more exogenous polynucleotides of interest comprise a polynucleotide encoding luciferase or fluorescent protein, a polynucleotide encoding a Fc receptor, a polynucleotide encoding an antibody, a polynucleotide encoding a cytokine, a polynucleotide encoding a protein having safety switch function, or any combination thereof.

7. The genetically-modified PSCs of claim 6, wherein the Fc receptor comprises CD16, CD64, or variants thereof, preferably non-cleavable CD16A shown in SEQ ID NO.: 3 and CD64/16A fusion protein shown in SEQ ID NO.: 5, and more preferably CD64/16A fusion protein shown in SEQ ID NO.: 5.

8. The genetically-modified PSCs of claim 6, wherein the cytokine comprises a membrane-bound cytokine, preferably a membrane-bound IL-15 and a variant thereof, and more preferably membrane-bound IL15RLI shown in SEQ ID NO.: 12.

9. The genetically-modified PSCs of claim 1, wherein the expression cassette further comprises one or more Ubiquitous Chromatin Opening Elements (UCOE) operably linked to the one or more promoters.

10. The genetically-modified PSCs of claim 9, wherein the expression cassette comprises the combination of EF1α promoter with 1550F shown in SEQ ID NO.: 14, or the combination of CMV promoter with 1550F shown in SEQ ID NO.: 14, and preferably the combination of EF1α promoter with 1550F shown in SEQ ID NO.: 14.

11. Genetically-modified iNK cells, comprising an expression cassette integrated at a selected locus, the expression cassette comprising one or more exogenous polynucleotides of interest, and one or more promoters operably linked to the one or more exogenous polynucleotides of interest, wherein the selected locus is CISH and/or Rosa26, and wherein the genetically-modified iNK cells have higher expression level and uniformity for the one or more exogenous polynucleotides of interest as compared with the iNK cells genetically modified under the same conditions except for the integration at a locus other than CISH and/or Rosa26.

12. The genetically-modified iNK cells of claim 11, wherein the genetically-modified iNK cells have at least two times and preferably at least four times higher expression uniformity for the one or more exogenous polynucleotides of interest than the iNK cells genetically modified under the same conditions except for the integration at the locus other than CISH and/or Rosa26.

13. The genetically-modified iNK cells of claim 11, wherein the locus other than CISH and/or Rosa26 is selected from AAVS1, CD38 and/or NKG2A.

14. The genetically-modified iNK cells of claim 11, wherein the one or more promoters are selected from EF1α, PGK, CAG, CMV, CLP, UBC and/or any promoter derived therefrom, and preferably EF1α, CMV, and/or CLP.

15. The genetically-modified iNK cells of claim 11, wherein the selected locus is CISH.

16. The genetically-modified iNK cells of claim 11, wherein the one or more exogenous polynucleotides of interest comprise a polynucleotide encoding luciferase or fluorescent protein, a polynucleotide encoding a Fc receptor, a polynucleotide encoding an antibody, a polynucleotide encoding a cytokine, a polynucleotide encoding a protein having safety switch function, or any combination thereof.

17. The genetically-modified iNK cells of claim 16, wherein the Fc receptor comprises CD16, CD64, or variants thereof, preferably non-cleavable CD16A shown in SEQ ID NO.: 3 and CD64/16A fusion protein shown in SEQ ID NO.: 5, and more preferably CD64/16A fusion protein shown in SEQ ID NO.: 5.

18. The genetically-modified iNK cells of claim 16, wherein the cytokine comprises a membrane-bound cytokine, preferably a membrane-bound IL-15 and a variant thereof, and more preferably membrane-bound IL15RLI shown in SEQ ID NO.: 12.

19. The genetically-modified iNK cells of claim 11, wherein the expression cassette further comprises one or more Ubiquitous Chromatin Opening Elements (UCOE) operably linked to the one or more promoters.

20. The genetically-modified iNK cells of claim 19, wherein the expression cassette comprises the combination of EF1α promoter with 1550F shown in SEQ ID NO.: 14, or the combination of CMV promoter with 1550F shown in SEQ ID NO.: 14, and preferably the combination of EF1α promoter with 1550F shown in SEQ ID NO.: 14.

21. The genetically-modified iNK cells of claim 20, wherein the genetically-modified iNK cells are immature or mature genetically-modified iNK cells, and preferably mature genetically-modified iNK cells.

22. A method for producing genetically-modified pluripotent stem cells (PSCs), comprising:
introducing into PSCs a construct comprising a site-specific endonuclease capable of introducing a double strand break at a selected locus of the genome of the PSCs and a construct comprising an expression cassette comprising one or more exogenous polynucleotides of interest and one or more promoters operably linked to the one or more exogenous polynucleotides of interest and a pair of homology arms specific to the selected locus and flanking the expression cassette; and
integrating the expression cassette comprising the one or more exogenous polynucleotides of interest and the one or more promoters into the genome of the PSCs at the selected locus via homologous recombination by the endonuclease to obtain the genetically-modified PSCs, wherein the selected locus is CISH and/or Rosa26;
wherein the genetically-modified PSCs are capable of differentiation into genetically-modified iNK cells having higher expression level and uniformity for the one or more exogenous polynucleotides of interest as compared with the PSCs genetically modified under the same conditions except for the integration at a locus other than CISH and/or Rosa26.

23. The method of claim 22, wherein the genetically-modified PSCs are capable of differentiation into genetically-modified iNK cells having at least two times and preferably at least four times higher expression uniformity for the one or more exogenous polynucleotides of interest than the PSCs genetically modified under the same conditions except for the integration at the locus other than CISH and/or Rosa26.

24. The method of claim 22, wherein the locus other than CISH and/or Rosa26 is selected from AAVS1, CD38 and/or NKG2A.

25. The method of claim 22, wherein the one or more promoters are selected from an exogenous promoter, and are directly operatively linked to the one or more exogenous polynucleotides of interest.

26. The method of claim 22, wherein the one or more promoters are selected from an endogenous promoter comprised in the selected locus, and are operatively linked to the one or more exogenous polynucleotides of interest upon the integration.

27. The method of claim 22, wherein the one or more promoters are selected from EF1α, PGK, CAG, CMV, CLP, UBC and/or any promoter derived therefrom, and preferably EF1α, CMV, and/or CLP.

28. The method of claim 22, wherein the selected locus is CISH.

29. The method of claim 22, wherein the one or more exogenous polynucleotides of interest comprise a polynucleotide encoding luciferase or fluorescent protein, a polynucleotide encoding a Fc receptor, a polynucleotide encoding an antibody, a polynucleotide encoding a cytokine, a polynucleotide encoding a protein having safety switch function, or any combination thereof.

30. The method of claim 29, wherein the Fc receptor comprises CD16, CD64, or variants thereof, preferably non-cleavable CD16A shown in SEQ ID NO.: 3 and CD64/16A fusion protein shown in SEQ ID NO.: 5, and more preferably CD64/16A fusion protein shown in SEQ ID NO.: 5.

31. The method of claim 29, wherein the cytokine comprises a membrane-bound cytokine, preferably a membrane-bound IL-15 and a variant thereof, and more preferably membrane-bound IL15RLI shown in SEQ ID NO.: 12.

32. The method of claim 22, wherein the expression cassette further comprises one or more Ubiquitous Chromatin Opening Elements (UCOE) operably linked to the one or more promoters.

33. The method of claim 32, wherein the expression cassette comprises the combination of EF1α promoter with 1550F shown in SEQ ID NO.: 14, or the combination of CMV promoter with 1550F shown in SEQ ID NO.: 14, and preferably the combination of EF1α promoter with 1550F shown in SEQ ID NO.: 14.

34. The method of claim 32, further comprising continuously expanding the genetically-modified PSCs.

35. The method of claim 34, wherein continuously expanding the genetically-modified PSCs comprises passaging the genetically-modified PSCs for at least passages and preferably at least 14 passages during the expansion.

36. A method for producing genetically-modified iNK cells, comprising producing genetically-modified PSCs according to the method of claim 22 and then differentiating the genetically-modified PSCs into NK cells, thereby producing the genetically-modified iNK cells,
wherein the genetically-modified iNK cells have higher expression level and uniformity for the one or more exogenous polynucleotides of interest as compared with the iNK cells genetically modified under the same conditions except for the integration at a locus other than CISH and/or Rosa26.

37. The method of claim 36, further comprising expanding and maturing the genetically-modified iNK cells.


SEQUENCE LISTING (79 sequences)

SEQ ID NO: 1 - protein, 627 aa, synthetic construct
  source 1..627: note = Antares2
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLWNGNKIIDERLINPDGSLLFRVTINGVTGWRLHERILARHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 2 - protein, 239 aa, synthetic construct
  source 1..239: note = GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - protein, 254 aa, synthetic construct
  source 1..254: note = non-cleavable CD16A with 158V/V mutation
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDK

SEQ ID NO: 4 - protein, 374 aa, synthetic construct
  source 1..374: note = CD64
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 5 - protein, 346 aa, synthetic construct
  source 1..346: note = CD64/16A
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHSFFPPGYQVSFCLVMVLLFAVDTGLYFSVKTNIRSSTRDWKDHKFKWRKDPQDK

SEQ ID NO: 6 - protein, 123 aa, synthetic construct
  source 1..123: note = NeoIL2
MDAMKRGLCCVLLLCGAVFVSPSPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 7 - protein, 229 aa, synthetic construct
  source 1..229: note = IL15RAsu
MDAMKRGLCCVLLLCGAVFVSPSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRD

SEQ ID NO: 8 - protein, 241 aa, synthetic construct
  source 1..241: note = IL15RLI
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 9 - protein, 265 aa, synthetic construct
  source 1..265: note = IL15ILR
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPP

SEQ ID NO: 10 - protein, 149 aa, synthetic construct
  source 1..149: note = IL15-TPA
MDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 11 - protein, 219 aa, synthetic construct
  source 1..219: note = mbIL15
MALPVTALLLPLALLLHAARPNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSAKPTTTPAPRPPTPAPTIASQPLSLRPEASRPAAGGAVHTRGLDFACDIYIWAPLAGTCGVLLLSLVIT

SEQ ID NO: 12 - protein, 325 aa, synthetic construct
  source 1..325: note = mbIL15-RLI
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSAKPTTTPAPRPPTPAPTIASQPLSLRPEASRPAAGGAVHTRGLDFACDIYIWAPLAGTCGVLLLSLVIT

SEQ ID NO: 13 - protein, 648 aa, synthetic construct
  source 1..648: note = mbIL21-mbIL15
MWLQSLLLLGTVACSISQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSESKYGPPCPPCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKAFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFCVGGTRGGGEGRGSLLTCGDVEENPGPRMALPVTALLLPLALLLHAARPNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSAKPTTTPAPRPPTPAPTIASQPLSLRPEASRPAAGGAVHTRGLDFACDIYIWAPLAGTCGVLLLSLVIT

SEQ ID NO: 14 - DNA, 1550 bp, synthetic construct
  source 1..1550: mol_type = other DNA; note = UCOE1550F
ggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgaccgtttctcacctgccttgcgccaaggcagggggcgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcc

SEQ ID NO: 15 - DNA, 1550 bp, synthetic construct
  source 1..1550: mol_type = other DNA; note = UCOE1550R
ggagacgccgtggcccccgaagcagcgtgctttagaaagggaataagaattcccgcctccgcgccccactttcaccccagcggggcagcgtccgccatgtgaaagctccccatcccccacccccagtgaagggaaatggcgccgggaggctgagggtggggaagctgtttgtacgctcaggcctccgctcaagaccccgttcataaaccttaagccccactgctactgaattggtccgatttcctgcctctctcccacggaggcggctggccgacttccactgaggcgccaacggcctcgccatgcccttttcaataactcattgatttcaaacccgttacctccatcgcggactcagtcgcttcagcccgatttcccgcagccgagcgagatgagagagatctccgcggacgaacacgaaccggactcgtcctggcgctgtagtgagaactgccgctgctcgagaaacaactctgcgaggagcacctccgcacgggacccggcgctgctgctactgccgctagagccgctgccgccgcttttctagaaccttcccccccactaacgcgtcttccgctacgtcaggccgtcgcgtaaacgccctatccgccgccaatggcgggaaggctctacgccccaccttacgccaaatgcgtactcctcccacccttgcggccagagacagtacccgacgttacttccgtaaatgcgctcaatgaattgcggaaggctagagtcctgctagttactacctcttggaatagggtcccgccccctgccttggcgcaaggcaggtgagaaacggtcgcgcagtttgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggcc

SEQ ID NO: 16 - DNA, 1194 bp, synthetic construct
  source 1..1194: mol_type = other DNA; note = UCOE1194F
ggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgaccgtttctcacctgccttgcgccaaggcagggggcgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgact

SEQ ID NO: 17 - DNA, 1194 bp, synthetic construct
  source 1..1194: mol_type = other DNA; note = UCOE1194R
agtcgcttcagcccgatttcccgcagccgagcgagatgagagagatctccgcggacgaacacgaaccggactcgtcctggcgctgtagtgagaactgccgctgctcgagaaacaactctgcgaggagcacctccgcacgggacccggcgctgctgctactgccgctagagccgctgccgccgcttttctagaaccttcccccccactaacgcgtcttccgctacgtcaggccgtcgcgtaaacgccctatccgccgccaatggcgggaaggctctacgccccaccttacgccaaatgcgtactcctcccacccttgcggccagagacagtacccgacgttacttccgtaaatgcgctcaatgaattgcggaaggctagagtcctgctagttactacctcttggaatagggtcccgccccctgccttggcgcaaggcaggtgagaaacggtcgcgcagtttgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggcc

SEQ ID NO: 18 - DNA, 458 bp, synthetic construct
  source 1..458: mol_type = other DNA; note = UCOE458F
gcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagc

SEQ ID NO: 19 - DNA, 458 bp, synthetic construct
  source 1..458: mol_type = other DNA; note = UCOE458R
gctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacacgcgtcccgggggcccggttctccgc

SEQ ID NO: 20 - DNA, 396 bp, synthetic construct
  source 1..396: mol_type = other DNA; note = UCOE396F
acagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctccc

SEQ ID NO: 21 - DNA, 396 bp, synthetic construct
  source 1..396: mol_type = other DNA; note = UCOE396R
gggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgt

SEQ ID NO: 22 - DNA, 761 bp, synthetic construct
  source 1..761: mol_type = other DNA; note = SRF6-3F
gcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaa

SEQ ID NO: 23 - DNA, 2401 bp, synthetic construct
  source 1..2401: mol_type = other DNA; note = cHS4
gggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggcagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgag

SEQ ID NO: 24 - DNA, 20 bp, synthetic construct
  source 1..20: mol_type = other DNA; note = gRNA-CISH Targeting Sequence 1
ttctagacctcgtcctttgc

SEQ ID NO: 25 - DNA, 20 bp, synthetic construct
  source 1..20: mol_type = other DNA; note = gRNA-CISH Targeting Sequence 2
gcacaggtgttgcaggctgc

SEQ ID NO: 26 - DNA, 20 bp, synthetic construct
  source 1..20: mol_type = other DNA; note = gRNA-CISH Targeting Sequence 3
gggttccattacggccagcg

SEQ ID NO: 27 - DNA, 20 bp, synthetic construct
  source 1..20: mol_type = other DNA; note = gRNA-CISH Targeting Sequence 4
gtggaggagcaggcagtgct

SEQ ID NO: 28 - DNA, 20 bp, synthetic construct
  source 1..20: mol_type = other DNA; note = gRNA-CISH Targeting Sequence 5
gccaaaggtgctggacccag

SEQ ID NO: 29 - DNA, 20 bp, synthetic construct
  source 1..20: mol_type = other DNA; note = gRNA-CISH Targeting Sequence 6
acatggtcctctgcgttcag

SEQ ID NO: 30 - DNA, 20 bp, synthetic construct
  source 1..20: mol_type = other DNA; note = gRNA-Rosa26 Targeting Sequence 1
gtttagagtcacatgcaaca

SEQ ID NO: 31 - DNA, 20 bp, synthetic construct
  source 1..20: mol_type = other DNA; note = gRNA-Rosa26 Targeting Sequence 2
tgatctcagacaatgaacct

SEQ ID NO: 32 - DNA, 20 bp, synthetic construct
  source 1..20: mol_type = other DNA; note = gRNA-Rosa26 Targeting Sequence 3
ttcttacctgagcatcccat

SEQ ID NO: 33 - DNA, 20 bp, synthetic construct
  source 1..20: mol_type = other DNA; note = gRNA-Rosa26 Targeting Sequence 4
ggatcgactattatatcata

SEQ ID NO: 34 - DNA, 20 bp, synthetic construct
  source 1..20: mol_type = other DNA; note = gRNA-Rosa26 Targeting Sequence 5
attggagccactgtacctac

SEQ ID NO: 35 - DNA, 20 bp, synthetic construct
  source 1..20: mol_type = other DNA; note = gRNA-Rosa26 Targeting Sequence 6
ggcgatgacgagatcacgcg

SEQ ID NO: 36 - DNA, 20 bp, synthetic construct
  source 1..20: mol_type = other DNA; note = gRNA-AAVS1 Targeting Sequence 1
tgcctcacctggcgatatct

SEQ ID NO: 37 - DNA, 20 bp, synthetic construct
  source 1..20: mol_type = other DNA; note = gRNA-AAVS1 Targeting Sequence 2
gtgcagggcgctgataccgt

SEQ ID NO: 38 - DNA, 20 bp, synthetic construct
  source 1..20: mol_type = other DNA; note = gRNA-AAVS1 Targeting Sequence 3
gtccctagtggccccactgt

SEQ ID NO: 39 - DNA, 20 bp, synthetic construct
  source 1..20: mol_type = other DNA; note = gRNA-AAVS1 Targeting Sequence 4
gccagcacctcgtgtcatga

SEQ ID NO: 40 - DNA, 20 bp, synthetic construct
  source 1..20: mol_type = other DNA; note = gRNA-AAVS1 Targeting Sequence 5
ggcactggccctcaccgcat

SEQ ID NO: 41 - DNA, 20 bp, synthetic construct
  source 1..20: mol_type = other DNA; note = gRNA-AAVS1 Targeting Sequence 6
caggctcagtacttcctcat

SEQ ID NO: 42 - DNA, 20 bp, synthetic construct
  source 1..20: mol_type = other DNA; note = gRNA-CD38 Targeting Sequence 1
tctggcccatcagttcacac

SEQ ID NO: 43 - DNA, 20 bp, synthetic construct
  source 1..20: mol_type = other DNA; note = gRNA-CD38 Targeting Sequence 2
tgagttcccaacttcattag

SEQ ID NO: 44 - DNA, 20 bp, synthetic construct
  source 1..20: mol_type = other DNA; note = gRNA-CD38 Targeting Sequence 3
ggccaactgcgagttcagcc

SEQ ID NO: 45 - DNA, 20 bp, synthetic construct
  source 1..20: mol_type = other DNA; note = gRNA-CD38 Targeting Sequence 4
catcacatggaccacatcac

SEQ ID NO: 46 - DNA, 20 bp, synthetic construct
  source 1..20: mol_type = other DNA; note = gRNA-CD38 Targeting Sequence 5
ctggaaaacggtttcccgca

SEQ ID NO: 47 - DNA, 20 bp, synthetic construct
  source 1..20: mol_type = other DNA; note = gRNA-CD38 Targeting Sequence 6
gatgtgcaagatgaatcctc

SEQ ID NO: 48 - DNA, 20 bp, synthetic construct
  source 1..20: mol_type = other DNA; note = gRNA-NKG2A Targeting Sequence 1
ggtctgagtagattactcct

SEQ ID NO: 49 - DNA, 20 bp, synthetic construct
  source 1..20: mol_type = other DNA; note = gRNA-NKG2A Targeting Sequence 2
tattattgaagatccacact

SEQ ID NO: 50 - DNA, 20 bp, synthetic construct
  source 1..20: mol_type = other DNA; note = gRNA-NKG2A Targeting Sequence 3
tccaacagttgttactacat

SEQ ID NO: 51 - DNA, 20 bp, synthetic construct
  source 1..20: mol_type = other DNA; note = gRNA-NKG2A Targeting Sequence 4
gctccagagaagctcattgt

SEQ ID NO: 52 - DNA, 20 bp, synthetic construct
  source 1..20: mol_type = other DNA; note = gRNA-NKG2A Targeting Sequence 5
ttctagctacattaatacag

SEQ ID NO: 53 - DNA, 20 bp, synthetic construct
  source 1..20: mol_type = other DNA; note = gRNA-NKG2A Targeting Sequence 6
atgggtgacaatgaatggtt

SEQ ID NO: 54 - DNA, 5959 bp, synthetic construct
  source 1..5959: mol_type = other DNA; note = pKI-Antares2 vector
cgatgccgatatcctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaacacgtggtcgcggccatctagagccaccatggtgtctaagggcgaggagctgattaaggagaatatgaggagtaagctgtatctggaagggagcgtcaacgggcatcagttcaagtgcacacacgagggcgagggcaagccttacgagggcaagcagaccaaccggatcaaggtggtggagggaggaccactgcctttcgcctttgacatcctggccacccacttcatgtatggcagcaaggtgtttatcaagtaccctgccgacctgccagattatttcaagcagtcctttccagagggcttcacatgggagcgcgtgatggtgttcgaggatggaggcgtgctgaccgcaacacaggacaccagcctgcaggatggcgagctgatctacaacgtgaaggtgcggggcgtgaactttcccgccaatggccctgtgatgcagaagaagaccctgggctgggagccttccaccgagacaatgtatccagcagacggaggcctggagggcagatgtgataaggccctgaagctggtgggaggaggacacctgcacgtgaatttcaagaccacatacaagagcaagaagccagtgaagatgcccggcgtgcactatgtggaccggagactggagaggatcaaggaggccgataacgagacatacgtggagcagtatgagcacgccgtggccagatactccaatctgggcggcggcttcacactggaggactttgtgggcgattggaggcagaccgccggctataacctgtctcaggtgctggagcagggaggcgtgagctccctgtttcagaatctgggcgtgtccgtgacccctatccagagaatcgtgctgtctggcgagaatggcctgaagatcgacatccacgtgatcatcccatacgagggcctgtccggcgatcagatgggccagatcgagaagatcttcaaggtggtgtaccccgtggacaaccaccacttcaaagtgatcctgcactatggcaccctggtcatcgacggcgtgaccccaaatatgatcgactacttcggcaggccctatgagggcatcgccgtgtttgatggcaagaagatcaccgtgacaggcaccctgtggaacggcaataagatcatcgacgagcgcctgatcaaccccgatggctctctgctgttccgggtgacaatcaatggagtgaccggatggaggctgcacgagagaatcctggccaggcacgagctgatcaaggagaacatgcgctctaagctgtacctggagggcagcgtgaatggccaccagtttaagtgtacccatgaaggcgagggcaagccctatgaaggcaagcagactaatcggatcaaggtcgtcgaaggaggaccactgcccttcgcctttgacatcctggcgacccacttcatgtacggctctaaagtgtttatcaagtatccagctgacctgccagattacttcaagcagagcttccctgagggctttacatgggagagagtgatggtgttcgaggacggaggcgtgctgacagcaacccaggacacctctctgcaggacggagagctgatctacaatgtgaaggtgaggggagtcaatttccctgctaacggacctgtgatgcagaagaagacactgggctgggagcccagcacagagacaatgtatcctgccgatggaggcctggagggccgctgtgacaaggccctgaagctggtcggcggcggacacctgcacgtgaacttcaagactacctacaagtccaagaagcctgtcaaaatgcctggagtccactatgtggacaggcgcctggagcggatcaaggaagctgataatgagacatatgtcgagcagtacgaacacgccgtcgcaaggtattctaatctgggaggagggatggatgaactgtataagggaagcggagctactaacttcagcctgctgaagcaggctggagacgtggaggagaaccctggacctaggatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaagatccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaagctcgcgtaggaattcggatcccgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctaacgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggtgctagcaccat

SEQ ID NO: 55 - DNA, 793 bp, synthetic construct
  source 1..793: mol_type = other DNA; note = Rosa26 5' homologous arm
aatggcgtattctggttggagtaaagctcctgtcagttacgccgtcgggagtacgcagccgcttagcgactctcgcgttgccccctgggtggggcgggtaggtaggtggggtgtagagatgctgggtgtgcgggcgcggccggcctcctgcggcgggaggggagggtcagtgaaatcggctctggcgcgggcgtcctcccaccctccccttccttcgggggagtcggtttacccgccgcctgcttgtcttcgacacctgattggctgtcgaagctgtgggaccgggcccttgctactggctcgagtctcacatgagcgaaaccactgcgcggggcgcgggggtggcggggaggcgggcgttggtacggtcctccccgaggccgagcgccgcagtgtctggccccgcgcccctgcgcaacgtggcaggaagcgcgcgctggaggcgggggcgggctgccggccgagacttctggatggcggcggccgcggctccgccccgggttcccaccgcctgaagggcgagacaagcccgacctgctacaggcactcgtgggggtgggggaggagcgggggtcggtccggctggtttgtgggtgggaggcgcttgttctccaaaaaccggcgcgagctgcaatcctgagggagctgcggtggaggaggtggagagaaggccgcacccttctgggcagggggaggggagtgccgcaatacctttatgggagttctctgctgcctcccgtcttgtaaggaccgccctgggcctggaagaagccctccctcctttcctcctcgc

SEQ ID NO: 56 - DNA, 457 bp, synthetic construct
  source 1..457: mol_type = other DNA; note = Rosa26 3' homologous arm
catgcctccatgtcgagtcgcttctcgattatgggcgggattcttttgcctaggcttaaggggctaacttggtccctgggcgttgccctgcaggggagtgagcagctgtaagatttgaggggcgactccgattagtttatcttcccacggactagagttggtgtcgaggttattgtaataagggtggggtagggaaatggagcttagtcattcacctggggctgattttatgcaacgagactgcggattatcactacttatcatttttggagcatttttctagagacagacataaagcatgatcacctgagttttataccatttgagacccttgctgcaccaccaaagtgtagcatcaggttaaatcttaatagaaaaattttagcttttgcttgagaaaccagtgcttccctccctcaccctctctccccaggctctctacccctttgcatcccta

SEQ ID NO: 57 - DNA, 817 bp, synthetic construct
  source 1..817: mol_type = other DNA; note = CISH 5' homologous arm
agtgtggtgagaacattagacatgcctatagggaatgatctgtgtttttggttgatataaaatagagagcaattaattactgtttagtaaatgcagggcagtggaaaagaatctttcaaaagaggatccccacatggaaacaatcaaggtggcttttgtgatgacaagtggggaacgagatactggactaactgagcccatggctgggtagaggggaagctgaggctcatagtgagatgggccagccagaggtcatgaaactagtctggggcataggggggtctctggagatgggggaagcatgggtcctccactcactgctcatccactgccttctagacctcgtcctttgctggctgtggagcggactgggcagcggcccctgtgggccccgtccctggaactgcccaagccagtcatgcagcccttgcctgctggggccttcctcgaggaggtggcagagggtaccccagcccagacagagagtgagccaaaggtgctggacccagaggaggatctgctgtgcatagccaagaccttctcctaccttcgggaatctggtgagtctgaggggggaggcaggccttttcctgagtagtctggtgggagaagcttagttctgcctttgagtctgatttgggaggcatagcccggcccaggagagtctgatgggagaggcacagccctctctagggaagcgtgaagctggaggcactgccttgacttgcactggattggttaccccagctaatttccactatgtcccttggccccctctgcacttgcctaggctggtattggggttccattacggcca

SEQ ID NO: 58 - DNA, 803 bp, synthetic construct
  source 1..803: mol_type = other DNA; note = CISH 3' homologous arm
gcgaggcccgacaacacctgcagaagatgccagaaggcacgttcttagtacgtgacagcacgcaccccagctacctgttcacgctgtcagtgaaaaccactcgtggccccaccaatgtacgcattgagtatgccgactccagcttccgtctggactccaactgcttgtccaggccacgcatcctggcctttccggatgtggtcagccttgtgcagcactatgtggcctcctgcactgctgatacccgaagcgacagccccgatcctgctcccaccccggccctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccgactacctccgacagtaccccttccagctctgactgtacggggcaatctgcccaccctcacccagtcgcaccctggaggggacatcagccccagctggacttgggcccccactgtccctcctccaggcatcctggtgcctgcatacctctggcagctggcccaggaagagccagcaagagcaaggcatgggagaggggaggtgtcacacaacttggaggtaaatgcccccaggccgcatgtggcttcattatactgagccatgtgtcagaggatggggagacaggcaggaccttgtctcacctgtgggctgggcccagacctccact

SEQ ID NO: 59 - DNA, 797 bp, synthetic construct
  source 1..797: mol_type = other DNA; note = AAVS1 5' homologous arm
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccaca

SEQ ID NO: 60 - DNA, 845 bp, synthetic construct
  source 1..845: mol_type = other DNA; note = AAVS1 3' homologous arm
gtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 61 - DNA, 801 bp, synthetic construct
  source 1..801: mol_type = other DNA; note = CD38 5' homologous arm
tgtgccagaccctgcttcaaacacattccatggactataaaattgcatctctgagcagctcctagagctggtagtaacaacttacatttactgggtgattaccatgtgccaggtattgtgctaaacacgttgtagatattaactcacttaatcctcgtaacaatcccatgaagtaggtactgctactatcccggctttacatctgaagtacagagaggttaagtaacttgccccatgtcatccagcaagaactaaatttgaacccagagcttagccactgatgcctcttgagagaaggagtcagacttaagttgagtctttaaaggtggttgaccaggcatttgtcagagttaagaaagagaggtaggacatccttttccaggcagagggcattgtgtgcacacacgtatagaagcaggcagcccaccctcatgctttccaggaagcaaatgtggctcaggtgtaaagtgcccggttgatgaagggagttagcggagggagtataaggatgtactgtctgcccccttaggacacctgcagaggattaaggtggctgtttctccctggaggtggagtgggtgggtcactgcacaggagcctatagttgttggtcttttaaactcttattggtgtaaccagccacggaactctgaggcaaggggttgggggtgggaagggaaacagagaaaaggcaagtgaaacagaaggggaggtgcagtttcagaacccagccagcctctctcttgctgcctagcctcctgccggcctcatcttcgcccagccaaccccgcctggagccct

SEQ ID NO: 62 - DNA, 794 bp, synthetic construct
  source 1..794: mol_type = other DNA; note = CD38 3' homologous arm
gcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgaggtgggttggcgactaaggcgcaccggtgggcactgcggggacagcagggccccgcgcgcagggaagccgcccggatcgcccggaaccgggcatcttccgtggcgggtcagccgagagcccgccgggtggtgctgagtagggagtcccgggctcggggctccgcgggccgctttcaggagcagctggccttggcaccgagcgtgcccgcgggaggcgggggggggcgctgctcggtggctctgctgcgtagccggtgaacacttggcaccgatgcccgccttctgggcaaggtgccctgagcccagcccctcgccgggctgcagcccaccctcggcgcgctcagcccgcttcaccgcttcagggacggaatagaactcgcagatgcagggtgtcgctgacattttcaactttttctgcggtttccgcccgctgtctctgacccgaaagtgcccccggacggttacagaggacacttaagtggtttgcaaagcctgtggtaggggaggagggtgtagaagggccaaaccacggaacttagttttattcatttatataaagcagcactccgattctttttgcg

SEQ ID NO: 63 - DNA, 743 bp, synthetic construct
  source 1..743: mol_type = other DNA; note = NKG2A 5' homologous arm
gtcaactttactattactcatgtgcagaccacatagtcttaaccattactggatttcaaaattaacataaaaatgagtgcagaagtttttctaatacaacattttaatttttaacacaggtaaaacatcagactttaagaaatatatttttattcctgaacttctttattccttagtaatttattgcttctcattgccccagcaataatattttgtcaaatgcagaaaatttatcttttttttttgagtcggagtctcactctgtcgcccaggctggagtgcagtggcacaatctccgctcactgcaacctctgccgcccatgttcaagagattctcctgcctcagcctcccgagtagctaggactacaggcgcctgacatcatgcccgactactttttgtatttttagtagagacggagtttcaccgtgttagccaggatggtctcgatctcctgacctcgtgatcggcatgcctcggcctcccaaagtgctgggattacaggcgtgagccaccgcgcccggcctaaaaatctttttttaaaacaaatattcataagaaacgtgtttaggcttgaagaaaatcagagaaagaactttagattatttaatgcaaaatgagctccaatactcgttctccacctcacccttttaattgcactagggaatcctgtatataaaccatttattaacttcttaactactgttattatagagtacagtccctgacatcacacactgcaga

SEQ ID NO: 64 - DNA, 794 bp, synthetic construct
  source 1..794: mol_type = other DNA; note = NKG2A 3' homologous arm
agtaatctactcagacctgaatctgcccccaaacccaaagaggcagcaacgaaaacctaaaggcaataaaaactccattttagcaactgaacaggaaataacctatgcggaattaaaccttcaaaaagcttctcaggattttcaagggaatgacaaaacctatcactgcaaaggtaaagcatttaaaagatcctcaatataacagtctaggatgtgcagcttggggtacaggaatgtggggaaagagaagggagtgctcatatatcttctatttgcaaagatcagaattccaagttgagatatgctatttcaatgtaaagtatgaagactgattgaactcattgttgaagtttgtagtctttgtcaaataattcatggagcattatttttcctgaaaattcaatggtatattattctgagaaaaagattacaatgggagatgagggtttggggtccaagtttctctgtatgattcctgtgcattcaggttctcttgtctgtgaatcttctaaacgactgtatccacctctcctttcgcactgttcccatttctctccctgcagatttaccatcagctccagagaagctcattgttgggatcctgggaattatctgtcttatcttaatggcctctgtggtaacgatagttgttattccctgtaagtctattttcgaagattacaaggggaattttcacgttaatgattgaatgtgcctctaaacatttcatattttcagggaatagagttctcattgtaatgtatatatttggactaaatgtggaatgattattc

SEQ ID NO: 65 - DNA, 82 bp, synthetic construct
  source 1..82: mol_type = other DNA; note = gRNA chimeric fragment
gttttagagctagaaatagcaagttaaaataaggctagtccgttatcaacttgaaaaagtggcaccgagtcggtgctttttt

SEQ ID NO: 66 - DNA, 4971 bp, synthetic construct
  source 1..4971: mol_type = other DNA; note = PB-PNEE vector
aattcgcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattacccgggagtcgagtaaagcgcaaatcttttttaaccctagaaagatagtctgcgtaaaattgacgcatgcattcttgaaatatcgctctctctttctaaatagcgcgaatccgtcgctgtgcatttaggacatctcagtcgccgcttggagctcccgtgaggcgtgtttgtcaatgcggtaagtgtcactgattttgaactataacaaccgcgtgagtcaaaatgacgcatgattatcttttacgtgacttttaagatttaactcatacgataattatattgttatttcgtgttctacttacgtgataacttattatatatatattttcttgttatagatatcgtgactcatcacactggcggccgcataacttcgtatagcatacattatacgaagttatcagccatcgatgccgatatcctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccatctagagcaactagtgggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaagcttaaatcccggcgcgccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaagcccggcattctgcacgcttcaaaagcgcacgtctgccgcgctgttctcctcttcctcatctccgggcctttcgacctgcagccgctagcatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaagatcttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgccatatgtgataacttcgtatagcatacattatacgaagttatgaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcag

SEQ ID NO: 67 - protein, 399 aa, synthetic construct
  source 1..399: note = CD16A-T2A-NeoIL2
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMDAMKRGLCCVLLLCGAVFVSPSPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 68 - protein, 505 aa, synthetic construct
  source 1..505: note = CD16A-T2A-IL15Rasu
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMDAMKRGLCCVLLLCGAVFVSPSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRD

SEQ ID NO: 69 - protein, 517 aa, synthetic construct
  source 1..517: note = CD16A-T2A-IL15RLI
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 70 - protein, 541 aa, synthetic construct
  source 1..541: note = CD16A-T2A-IL15ILR
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPP

SEQ ID NO: 71 - protein, 425 aa, synthetic construct
  source 1..425: note = CD16A-T2A-IL15-TPA
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 72 - protein, 495 aa, synthetic construct
  source 1..495: note = CD16A-T2A-mbIL15
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMALPVTALLLPLALLLHAARPNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSAKPTTTPAPRPPTPAPTIASQPLSLRPEASRPAAGGAVHTRGLDFACDIYIWAPLAGTCGVLLLSLVIT

SEQ ID NO: 73 - protein, 601 aa, synthetic construct
  source 1..601: note = CD16A-T2A-mbIL15-RLI
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSAKPTTTPAPRPPTPAPTIASQPLSLRPEASRPAAGGAVHTRGLDFACDIYIWAPLAGTCGVLLLSLVIT

SEQ ID NO: 74 - protein, 924 aa, synthetic construct
  source 1..924: note = CD16A-T2A-mbIL21-mbIL15
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDKGGGEGRGSLLTCGDVEENPGPRMWLQSLLLLGTVACSISQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSESKYGPPCPPCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKAFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFCVGGTRGGGEGRGSLLTCGDVEENPGPRMALPVTALLLPLALLLHAARPNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSAKPTTTPAPRPPTPAPTIASQPLSLRPEASRPAAGGAVHTRGLDFACDIYIWAPLAGTCGVLLLSLVIT

SEQ ID NO: 75 - protein, 491 aa, synthetic construct
  source 1..491: note = CD64/16A-T2A-NeoIL2
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHSFFPPGYQVSFCLVMVLLFAVDTGLYFSVKTNIRSSTRDWKDHKFKWRKDPQDKGGGEGRGSLLTCGDVEENPGPRMDAMKRGLCCVLLLCGAVFVSPSPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 76 - protein, 517 aa, synthetic construct
  source 1..517: note = CD64/16A-T2A-IL15-TPA
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHSFFPPGYQVSFCLVMVLLFAVDTGLYFSVKTNIRSSTRDWKDHKFKWRKDPQDKGGGEGRGSLLTCGDVEENPGPRMDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 77 - protein, 609 aa, synthetic construct
  source 1..609: note = CD64/16A-T2A-IL15RLI
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHSFFPPGYQVSFCLVMVLLFAVDTGLYFSVKTNIRSSTRDWKDHKFKWRKDPQDKGGGEGRGSLLTCGDVEENPGPRMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 78 - protein, 693 aa, synthetic construct
  source 1..693: note = CD64/16A-T2A-mbIL15RLI
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHSFFPPGYQVSFCLVMVLLFAVDTGLYFSVKTNIRSSTRDWKDHKFKWRKDPQDKGGGEGRGSLLTCGDVEENPGPRMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSGGGGSAKPTTTPAPRPPTPAPTIASQPLSLRPEASRPAAGGAVHTRGLDFACDIYIWAPLAGTCGVLLLSLVIT

SEQ ID NO: 79 - protein, 237 aa, synthetic construct
  source 1..237: note = mb PDL1 Nb
MALPVTALLLPLALLLHAARPQVQLQESGGGLVQPGGSLRLSCAASGFTFSSFAMSWVRQAPGKGLEWVSDIDTTGRTTDYADSVKGRFTISRDNAENTLYLQMNDLKPEDTAVYYCANVPKELVLSFGSWGPGTQVTVSSTRANWSHPQFEKGGGGSGGGGSGGGGSAKPTTTPAPRPPTPAPTIASQPLSLRPEASRPAAGGAVHTRGLDFACDIYIWAPLAGTCGVLLLSLVIT